(19) United States
(12) Patent Application Publication
(10) Pub. No.: US 2021/0346484 A1
(43) Pub. Date: Nov. 11, 2021

(54) METHOD OF PRODUCING A VACCINE COMPOSITION AND USES THEREOF

(71) Applicant: Medizinische Universitaet Wien, Vienna (AT)

(72) Inventors: Ursula Wiedermann-Schmidt, Vienna (AT); Joshua Tobias, Vienna (AT)

(21) Appl. No.: 16/770,871

(22) PCT Filed: Dec. 11, 2018
(86) PCT No.: PCT/IB2018/001489

§ 371 Date: Jun. 8, 2020

(30) Foreign Application Priority Data
Dec. 11, 2017 (AU) 2017904978

(51) Int. Cl. A61K 39/00; A61K 45/06; C07K 16/32; C07K 16/28

(52) U.S. Cl. CPC A61K 39/00116; A61K 45/06; C07K 16/32; A61K 38/00; C07K 16/2878; C07K 16/2818; C07K 16/2827; C07K 16/2875

(57) ABSTRACT

The present invention provides a method of producing a vaccine composition for the treatment of cancer comprising a fusion protein of at least two peptide sequences, the method comprising: (a) generating a library of peptide sequences; (b) obtaining at least one antibody that binds to an antigen associated with cancer or a checkpoint antigen; (c) screening the library generated in step (a) with the at least one antibody of step (b) to identify at least two peptide sequences that specifically bind to the at least one antibody; and (d) combining at least two of the peptide sequences identified in step (c) to produce a fusion protein, wherein the fusion protein, when administered to a subject, induces an antibody response directed against the antigen associated with cancer or the checkpoint antigen.

### TECHNICAL FIELD

The present invention relates generally to a multilevel approach for the development of a vaccine composition for the treatment of cancer.

### BACKGROUND

The leading cause of death worldwide is cancer, accounting for 8.2 million deaths in 2012 (World Cancer Report 2014). Although the disease is highly prominent in the developed world, almost 50% of breast cancer cases and 58% of deaths occur in less developed countries. A particularly frequent cancer is breast cancer, with an incidence of 1.3 million per year, bearing a life time risk of 1:8 and being responsible for around 380,000 deaths (Bray et al., 2004). Around 400,000 new cancer cases are diagnosed in Europe, and more than 130,000 deaths are reported every year (Ladjemi et al., 2010).

Currently, the most common forms of treating cancer involve surgery, chemical intervention and/or radiotherapy. Unless the cancer is restricted to a defined area, surgery alone cannot eliminate the cancer. Moreover, radiation treatment as well as chemotherapy may entail severe negative side effects. In view of the disadvantages associated with the current therapies, attempts have been made to find additional approaches for treating proliferative disorders, such as breast cancer, including immunotherapy.

Some cancers are characterised by or associated with the overexpression of certain antigens on the surface of the cancer cell. For instance, the clinical implications of Her2/neu over-expression in tumors have made Her2/neu an attractive target for antibody-mediated immunotherapy, alone or as an adjunct to conventional chemotherapy. The monoclonal antibody 4D5, for instance, has been shown to reduce the growth of Her2/neu expressing tumours in mice by direct and indirect mechanisms such as apoptosis, antibody-dependent cell-mediated cytotoxicity (ADCC) or complement-dependent cytotoxicity (CDC). Based on these results, a humanized form of this antibody, Trastuzutnab (Herceptin®), was tested in clinical trials. Increased overall survival of patients with breast tumors overexpressing Her2/neu was observed following cytotoxic treatment plus Herceptin®, as compared to chemotherapy or Trastuzumab alone. Herceptin® is now used as monotherapy but shows even higher efficacy in combination with cytotoxic chemotherapy. It is to be noted, however, that Trastuzumab is generally only effective in breast cancer where the Her2/neu receptor is overexpressed. Furthermore, multiple infusions are typically required, resulting in high treatment costs.

An alternative approach to the treatment or prevention of Her2/neu-associated cancers using passive immunotherapy with monoclonal antibodies such as Trastuzumab is based on the induction of tumour-specific humoral and/or cellular immune responses and the identification of antigens recognized by human B- and T-lymphocytes. For example, numerous antibodies directed against the extracellular domain (ECD) of Her2/neu have been generated by immunizing mice with cells expressing Her2/neu. The biological effect of these antibodies appears to be epitope-specific; that is, it is based on specific recognition of a short subsequence within the Her2/neu ECD. However, some antibodies have no effect or even actively stimulate tumour growth.

The use of individual fragments of the extracellular domain (ECD) of Her2/neu, including B cell epitopes, has also been approached as a potential vaccine. For example, WO 2002/068474 describes a vaccine that comprises a peptide of 9-25 amino acids which sequence occurs in the extracellular part of the Her2/neu protein. Further, WO 2007/118660 describes a multi-peptide vaccine comprising a specific combination of peptides presenting different amino acid sequences as occur in the extracellular part of the Her2/neu protein. These peptides may be administered individually or in the form of multiple discrete peptides, each preferably conjugated separately to a delivery system. In yet another example, WO 2011/020604 describes fusion peptides comprising multiple Her2/neu B cell epitopes coupled to a virosome delivery system. These virosomes were shown to a induce a higher antibody titre against a single B cell epitope as compared to the same fusion peptides formulated with Montanide™ or an ISCOM-based delivery system. In yet a further example, WO 2001/078766 describes a vaccine composition comprising one or more mimotopes individually conjugated to a macromolecular carrier. Rabbits that were immunised with the vaccine composition were shown to produce antibodies that bound to the native Her2/neu protein.

However, despite several attempts to develop a suitable vaccine for inducing immunity against cancer antigens, there is still no effective vaccine in clinical use. It is an aim of the present invention to solve or partly alleviate these problems by providing an improved, multilevel approach to the development of a vaccine composition for the treatment of cancer and the use of such vaccine compositions for treating a cancer.

### SUMMARY OF THE INVENTION

In an aspect of the present disclosure, there is provided a method of producing a vaccine composition for the treatment of cancer comprising a fusion protein of at least two peptide sequences, the method comprising:
(a) generating a library of peptide sequences;
(b) obtaining at least one antibody that binds to an antigen associated with cancer and/or a checkpoint antigen;
(c) screening the library generated in step (a) with the at least one antibody of step (b) to identify at least two peptide sequences that specifically bind to the at least one antibody; and
(d) combining at least two of the peptide sequences identified in step (c) to produce a fusion protein,
   wherein the fusion protein, when administered to a subject, induces an antibody response directed against the antigen associated with cancer and/or the checkpoint antigen.

### BRIEF DESCRIPTION OF THE FIGURES

FIG. 1 is a diagrammatic representation showing HER-2/neu which, in contrast to the other family members, is constitutively in an extended configuration.

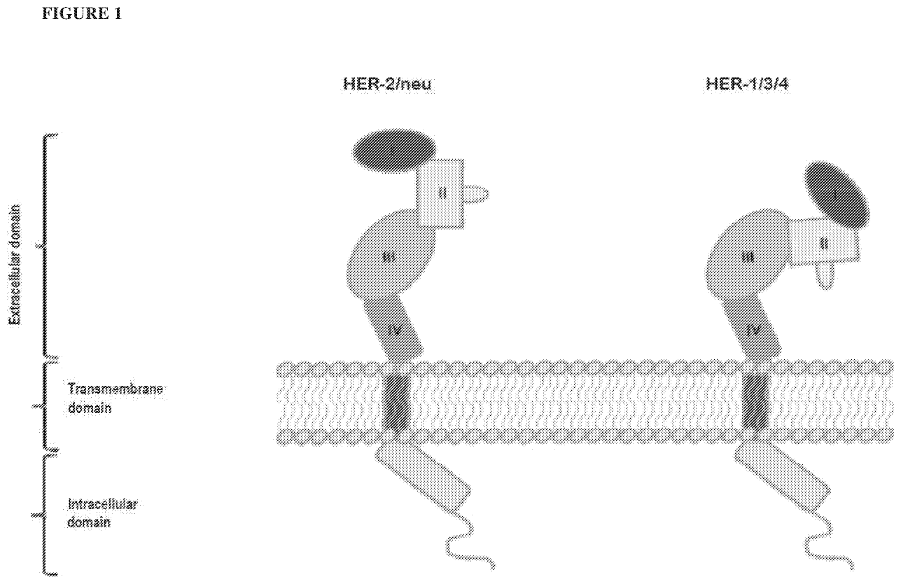

FIG. 2 is a diagrammatic illustration of the PepID technology for producing and identifying overlapping peptide fragments.

FIG. 3 shows a colony blot of a peptide library comprising 15-mer peptides of the extracellular domain (ECD) of Her-2, offset by 3 amino acids.

FIG. 4 shows the structure of the extracellular domain of Her-2 complexed with the Fab portion of the anti-Her-2 antibody, Trastuzumab (from Cho et al., 2003, Nature; 421(6924):756-60).

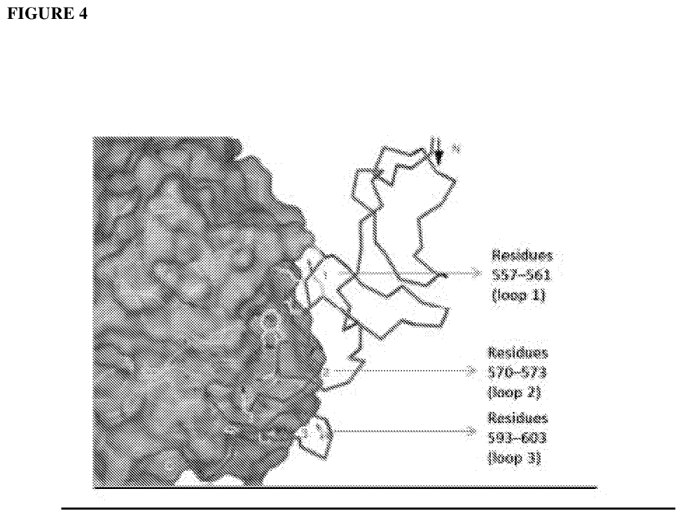

FIG. 5 is an illustration of the identified mimotopes position on Her-2/neu extracellular subdomains.

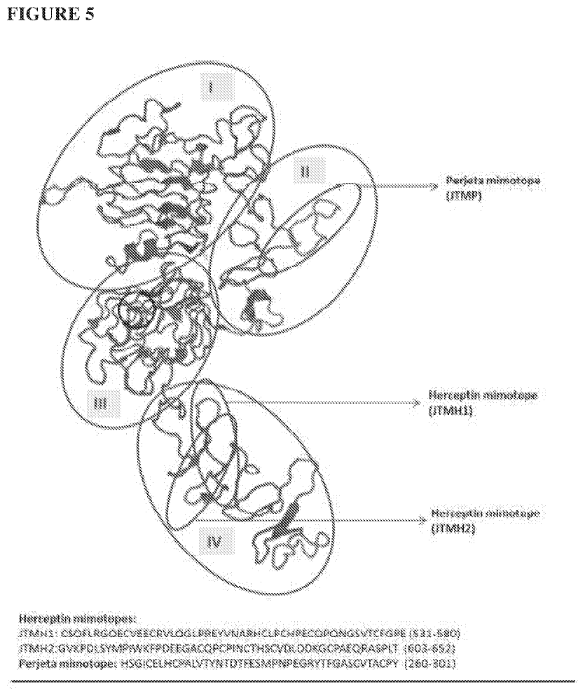

FIG. 6 shows the level of anti-Her-2 IgG antibodies generated in mice immunized with JMTP conjugated to CRM197 (10 μg; formulated in the presence of Alum; n=3); JTMH1 conjugated to CRM197 (10 μg; formulated in the presence of Alum; n=6); JTMH2 conjugated to CRM197 (10 μg; formulated in the presence of Alum; n=6); JTMH1+JTMH2 (5-10 μg; each conjugated to CRM197 and formulated in the presence of Alum; n=6); JMTP+JTMH2 (5-10 μg; each conjugated to CRM197 and formulated in the presence of Alum; n=6); JMTP+JTMH2 (5-10 μg; each conjugated to CRM197 and formulated in the presence of Alum; n=6). The level of antibodies was measured by ELISA.

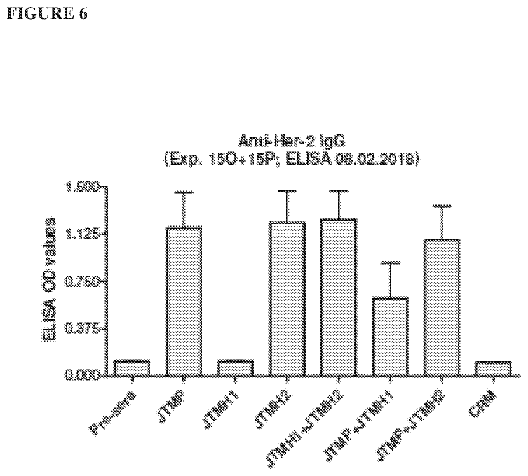

FIG. 7 shows levels of 1L-2 and interferon-gamma (IFN-γ) by splenocytes from immunized mice and exposed to JMTP, JTMH1 or JTMH2, alone or conjugated to CRM197. The levels of secreted IL-2 and IFNγ were measured by ELISA.

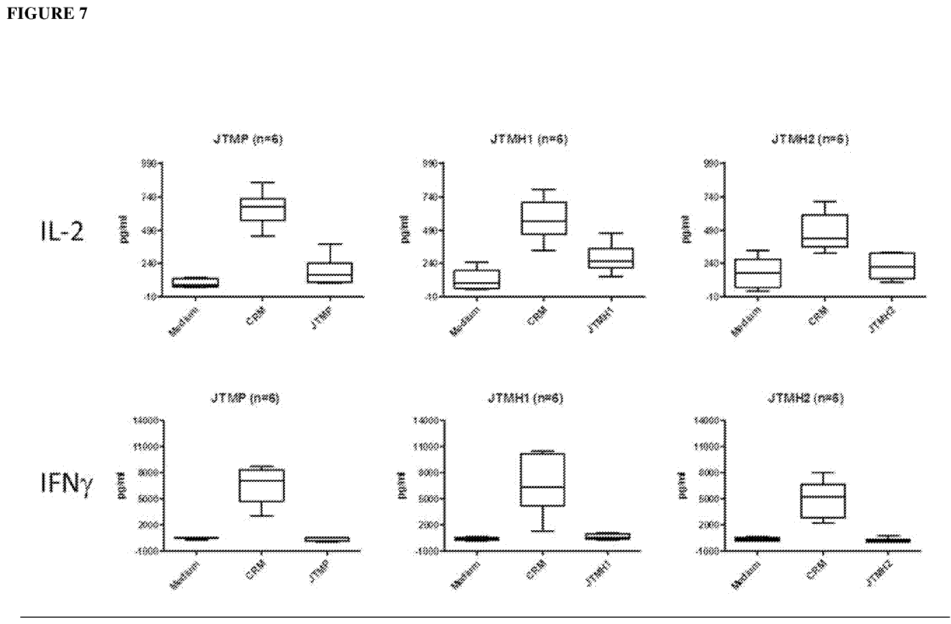

FIG. 8 shows the level of anti-JTMP, anti-JTMH2 and anti-Her-2/neu IgG antibodies in female FVB/N mice transgenic for the activated rat c-neu oncogene (MMTV-c-neu). Mice were immunized with JMTP conjugated to CRM197 (10 μg; formulated in the presence of Alum; n=3); JTMH1 conjugated to CRM197 (10 μg; formulated in the presence of Alum; n=6); JTMH2 conjugated to CRM197 (10 μg; formulated in the presence of Alum; n=6). The level of antibodies was measured by ELISA.

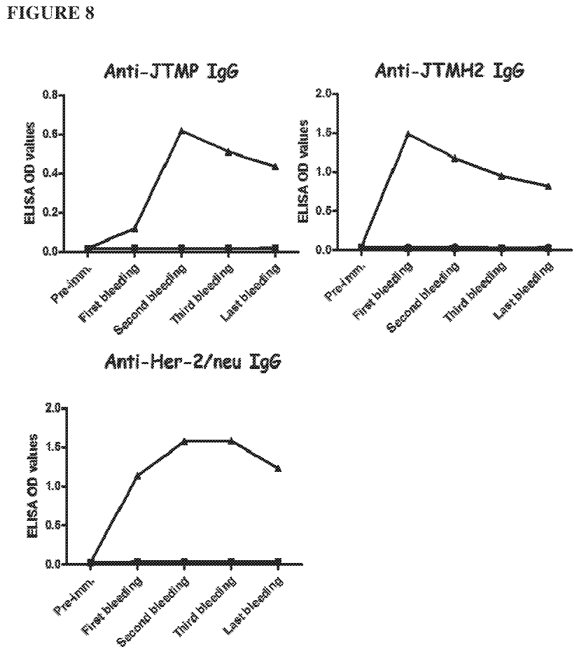

FIG. 9 shows the level of anti-Her-2 IgG antibodies generated in mice immunized with CRM197 alone (10 μg; formulated in the presence of Alum; n=6) or with a combination of JMTP+JTMH2 (10 μg; each conjugated to CRM197 and formulated in the presence of Alum; n=7). Naïve controls (n=8) received saline alone. The level of antibodies was measured by ELISA.

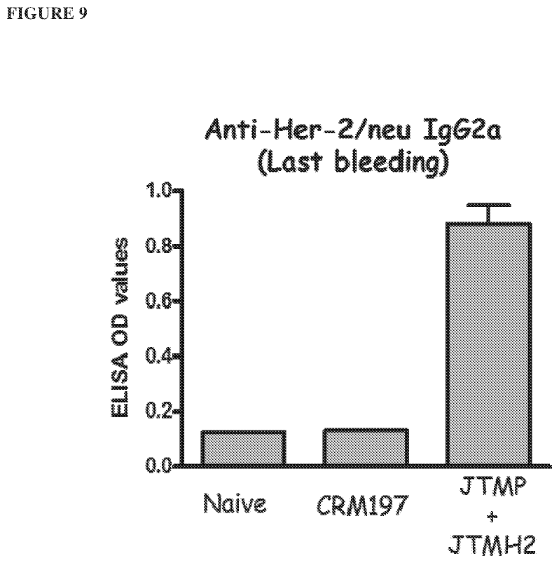

FIG. 10 shows the survival rate of transgenic female FVB/N mice (MMTV-c-neu) immunised with CRM197 alone (10 μg; formulated in the presence of Alum; n=6) or with a combination of JMTP+JTMH2 (10 μg; each conjugated to CRM197 and formulated in the presence of Alum; n=7). Naïve controls (n=8) received saline alone.

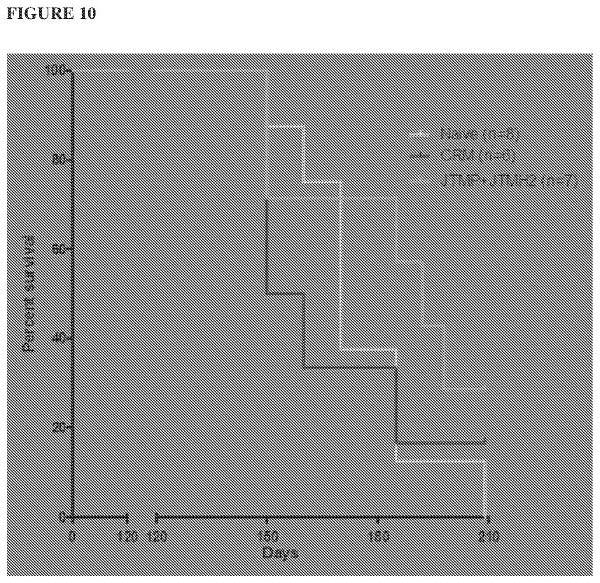

FIG. 11 shows the growth rate of tumours in transgenic female FVB/N mice (MMTV-c-neu) immunised with CRM197 alone (10 μg; formulated in the presence of Alum; n=5) or with a combination of JMTP+JTMH2 (10 μg; each conjugated to CRM197 and formulated in the presence of Alum; n=6). Naïve controls (n=8) received saline alone.

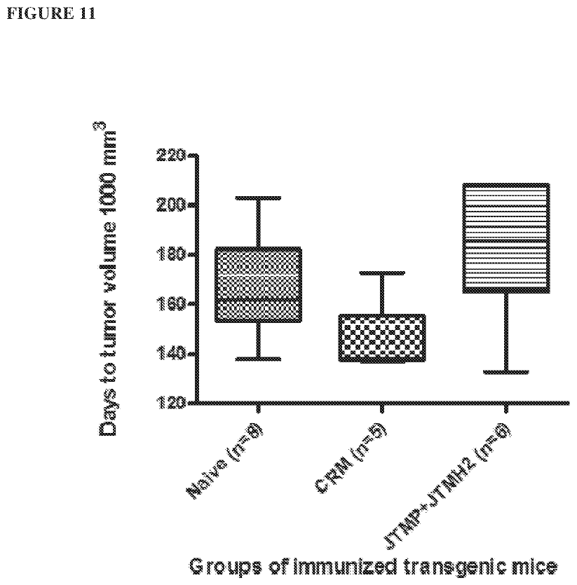

### DETAILED DESCRIPTION

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention. Practitioners may refer to Sambrook et al., (1989) Molecular Cloning: A Laboratory Manual, 2nd ed., Cold Spring Harbor Press, Plainsview, N.Y., and Ausubel et al. (1999) Current Protocols in Molecular Biology (Supplement 47), John Wiley & Sons, New York, Murphy et al. (1995) Virus Taxonomy Springer Verlag:79-87, for definitions and terms of the art and other methods known to the person skilled in the art.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a peptide” includes a single peptide, as well as two or more peptides; reference to “an epitope” includes one epitope, as well as two or more epitopes, and so forth.

Nucleotide and amino acid sequences are referred to by sequence identifier numbers (SEQ ID NOs:), as shown in Table 1, below. The SEQ ID NOs: correspond numerically to the sequence identifiers <400>1, <400>2, etc. A table of sequence identifiers is provided herein.

TABLE 1

| | SEQ ID |
|---|---|
| Amino acid sequences | NO: |
| GADDVVDSSKSFVMENFSSYHGTKPGYV | 1 |
| DSIQKGIQKPKSGTQGNYDDDWKEFYSTD | |
| NKYDAAGYSVDNENPLSGKAGGVVKVTY | |
| PGLTKVLALKVDNAETIKKELGLSLTEPL | |
| MEQVGTEEFIKRFGDGASRVVLSLPFAEGS | |
| SSVEYINNWEQAKALSVELEINFETRGKR | |
| GQDAMYEYMAQACAGNRVRRSVGSSLSC | |
| INLDWDVIRDKTKTKIESLKEHGPIKNKMS | |
| ESPNKTVSEEKAKQYLEEFHQTALEHPELS | |
| ELKTVTGTNPVFAGANYAAWAVNVAQVI | |
| DSETADNLEKTTAALSILPGIGSVMGIADG | |
| AVHHNTEEIVAQSIALSSLMVAQAIPLVGE | |
| LVDIGFAAYNFVESIINLFQVVHNSYNRPA | |
| YSPGHKTQPFLHDGYAVSWNTVEDSIRRT | |
| GFQGESGHDIKITAENTPLPIAGVLLPTIPG | |
| KLDVNKSKTHISVNGRKIRMRCRAIDGDV | |
| TFCRPKSPVYVGNGVHANLHVAFHRSSSE | |
| KIHSNEISSDSIGVLGYQKTVDHTKVNSKL | |
| SLFFEIKS | |

The present disclosure is predicated, at least in part, on the inventors' multilevel approach to the development of a vaccine composition for the treatment of a cancer, an approach can be adopted for any cancer that is characterised by, or associated with, an antigen, whether the cancer is characterised by, or associated with, the overexpression of the antigen, or is characterised by, or associated with, aberrant activity of the antigen.

Antigens targeted for therapy include tumour-associated antigens and checkpoint proteins.

Thus, in an aspect of the present disclosure, there is provided a method of producing a vaccine composition for the treatment of cancer comprising a fusion protein of at least two peptide sequences, the method comprising:
(a) generating a library of peptide sequences;
(b) obtaining at least one antibody that binds to an antigen associated with cancer and/or a checkpoint antigen;
(c) screening the library generated in step (a) with the at least one antibody of step (b) to identify at least two peptide sequences that specifically bind to the at least one antibody; and
(d) combining at least two of the peptide sequences identified in step (c) to produce a fusion protein,
   wherein the fusion protein, when administered to a subject, induces an antibody response directed against the antigen associated with cancer and/or the checkpoint antigen.

The terms “antigen associated with cancer,” tumour-associated antigen”, “tumour antigen”, “cancer antigen” and the like are used interchangeably herein to mean an antigen that is aberrantly expressed in cancer cells or tissue. In some embodiments, the antigen that are expressed under normal conditions in a limited number of tissues and/or organs or in specific developmental stages. For example, the antigen may be specifically expressed under normal conditions in stomach tissue and is expressed or aberrantly expressed in one or more cancer cells. The expression of antigen is reactivated in cancer cells or tissue irrespective of the origin of the cancer. In some embodiments, the antigen includes differentiation antigens, preferably cell type-specific differentiation antigens (i.e., proteins that are specifically expressed under normal conditions in a certain cell type at a certain differentiation stage), cancer/testis antigens (i.e., proteins that are specifically expressed under normal conditions in testis and sometimes in placenta), and germline specific antigens. In an embodiment, the antigen associated with cancer is expressed on the cell surface of a cancer cell and is preferably not or only rarely expressed on normal cells and tissues. Preferably, the antigen or the aberrant expression of the antigen identifies cancer cells, preferably tumour cells. In some embodiments, the antigen that is expressed by a cancer cell in a subject (e.g., a patient suffering from cancer) is a self-protein. It will be understood, however, that no autoantibodies directed against the antigen are typically found in a detectable level under normal conditions in a subject carrying the antigen (typically a healthy patient that does not have cancer) or such autoantibodies can only be found in an amount below a threshold concentration that would be necessary to damage the tissue or cells carrying the antigen.

In some embodiments, the amino acid sequence of the antigen associated with cancer is identical between the antigen as it is expressed in normal tissues and the antigen as it is expressed in cancer cells or tissue. In other embodiments, the antigen comprises a mutation in its amino acid sequence when compared to the amino acid sequence of the antigen as it would otherwise be expressed in normal tissue or cells.

The terms “normal tissue” or “normal conditions” typically refer to healthy tissue or the conditions in a healthy subject; that is, non-pathological conditions, wherein “healthy” preferably means non-tumorigenic or non-cancerous.

The term “specifically expressed” typically means that the antigen is only, or predominantly, expressed in a specific tissue or organ. For example, a antigen specifically expressed in breast tissue means that the antigen is primarily expressed in breast tissue and is not expressed in other tissues or is not expressed to a significant extent in other tissue or organs. Thus, an antigen that is exclusively expressed in cells of breast tissue and to a significantly lesser extent in any other tissue, such as the gastric mucosa, is specifically expressed in cells of breast tissue. In some embodiments, the antigen may also be specifically expressed under normal conditions in more than one tissue type or organ (e.g., 2, 3, 4, 5, 6 or more) tissue types or organs. For example, if an antigen associated with cancer is expressed under normal conditions to an approximately equal extent in breast and stomach tissue, the antigen is considered to be specifically expressed in breast and stomach tissue.

As used herein, the term “self protein” means a protein that is encoded by the genome of the subject and that is under normal conditions (i.e., non-pathological conditions) optionally expressed in certain normal tissue types or at certain stages of development.

The terms “cancer” and “tumour” are used interchangeably herein and will be understood as comprising malignant cells; also referred to as neoplastic cells, cancer cells or tumour cells. By “malignant cell” is meant an abnormal cell that grows by uncontrolled cellular proliferation and will typically continues to grow after the initial growth stimulus has ceased.

In some embodiments, the cancer is characterized by the presence of malignant cells in which the antigen associated with the cancer (e.g., a tumor-associated antigen) is expressed or aberrantly expressed (e.g., overexpressed) by the malignant cells or tissue. Preferably, the cancer is characterized by surface expression of the antigen associated with cancer. By “aberrant” or “abnormal” expression is meant that expression of the antigen associated with cancer is altered, preferably increased, compared to the state in a non-malignant or normal cell or in a healthy individual (i.e., in an individual not having a disease associated with aberrant or abnormal expression of the antigen). In some embodiments, the increase in expression of the antigen refers to an increase by at least 10%, preferably by at least 20%, preferably by at least 50%, preferably by at least 100%, or more. In some embodiment, aberrant or abnormal expression of the antigen associated with cancer means that the antigen is only detectable on the cancer cells or tissue, while expression in normal or healthy tissue is undetectable.

It will be understood that the methods described herein are applicable to producing a vaccine composition for the treatment of any cancer, as long as the cancer is considered treatable by a targeted antibody response induced by the fusion protein. Illustrative examples of suitable cancers include leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, and lung cancer, lung carcinomas, prostate carcinomas, colon carcinomas, renal cell carcinomas, cervical carcinomas and the metastases thereof.

Suitable antigens associated with cancer will be known to persons skilled in the art. In an embodiment, the antigen associated with cancer is selected from the group consisting of EGFR (e.g., Her2/neu, Her-1), BAGE (B melanoma antigen), CEA (carcinoembryonic antigen), CpG (cytosine-phosphate diesterguanine), Gp100 (glycoprotein 100), h-TERT (telomerase transcriptase), MAGE (melanoma antigen-encoding gene), Melan-A (melanoma antigen recognized by T cells) and MUC-1 (mucin-1). However, it will also be understood that the choice of antigen that is to be the target of the vaccine composition produced by the methods disclosed herein will typically depend on the intended use of the vaccine composition. For example, if the vaccine composition is intended to treat subjects with breast cancer, then the antigen will typically be an antigen that is associated with (e.g., overexpressed by) the breast cancer. Suitable examples of antigens associated with breast cancer will be familiar to persons skilled in the art, illustrative examples of which include the epidermal growth factor receptors Her2/neu and Her1.

As used herein, the term “checkpoint antigen” typically means an antigen that is involved in endogenous inhibitory pathways for immune system function, such as those that act to maintain self-tolerance and modulate the duration and extent of immune response to antigenic stimulation. Studies have shown, however, checkpoint antigens reduce the effectiveness of a host's immune response towards the cancer, resulting in tumour growth (see Nirschl & Drake, 2013, Clin Cancer Res 19:4917-24).

Suitable checkpoint antigens will be familiar to persons skilled in the art, illustrative examples of which are discussed by Pardoll (2012, Nature Reviews Cancer 12:252-64). Other illustrative examples of suitable checkpoint antigens include CTLA-4 (cytotoxic T lymphocyte antigen-4) and its ligands CD80 and CD86; PD1 (programmed cell death protein 1), PD-L1 (programmed cell death ligand 1), PD-L2 (programmed cell death ligand 2), LAG-3 (lymphocyte activation gene-3) and its ligand MHC class I or II, TIM-3 (T cell immunoglobulin and mucin protein-3) and its ligand GAL-9, B- and T-lymphocyte attenuator (BTLA) and its ligand herpes virus entry mediator (HVEM) and several others, as discussed, for example, by Nirschl & Drake (2013 Clin Cancer Res 19:4917-24). In an embodiment disclosed herein, the checkpoint antigen is PD1.

As described elsewhere herein, the methods disclosed herein can also be used to produce a vaccine composition for targeting multiple antigens associated with cancer and/or checkpoint antigens. For example, in an embodiment disclosed herein, the vaccine composition produced by the methods disclosed herein is designed such that the fusion protein, when administered, induces an antibody response directed against an antigen associated with cancer and a checkpoint antigen. In another embodiment disclosed herein, the vaccine composition produced by the methods disclosed herein is designed such that the fusion protein, when administered, induces an antibody response directed against more than one antigen associated with cancer (e.g., 2, 3, 4, 5, 6, 7 or more antigens associated with cancer). In yet another embodiment, the vaccine composition produced by the methods disclosed herein is designed such that the fusion protein, when administered, induces an antibody response directed against more than one checkpoint antigen (e.g., 2, 3, 4, 5, 6, 7 or more checkpoint antigens). In a further embodiment, the vaccine composition produced by the methods disclosed herein is designed such that the fusion protein, when administered, induces an antibody response directed against more than one antigen associated with cancer (e.g., 2, 3, 4, 5, 6, 7 or more antigens associated with cancer) and more than one checkpoint antigen (e.g., 2, 3, 4, 5, 6, 7 or more checkpoint antigens).

In an embedment of the methods disclosed herein, the fusion protein, when administered to a subject, induces an antibody response directed against the antigen associated with cancer and the checkpoint antigen.

### Generating a Library of Peptide Sequences

The terms “peptide” and “polypeptide” are used herein in their broadest sense to refer to a molecule of two or more amino acid residues, or amino acid analogs. The amino acid residues may be linked by peptide bonds, or alternatively by other bonds, e.g. ester, ether etc., but in most cases will be linked by peptide bonds.

As used herein, the terms “amino acid” or “amino acid residue” encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine.

In an embodiment, the library of peptide sequences generated in step (a) comprises fragments of the antigen associated with the cancer, as herein described. In an embodiment, the library of peptide sequences generated in step (a) comprises fragments of the checkpoint antigen, as herein described.

In an embodiment, the library of peptide sequences generated in step (a) comprises at least two peptide sequences (e.g., 2, 3, 4, 5, 6, 7, 8, 9, 10, 11 or more peptide sequences). Thus, in an embodiment, the library of peptide sequences generated in step (a) comprises at least 5 peptide sequences, preferably at least 10 peptide sequences, preferably at least 20 peptide sequences, preferably at least 25 peptide sequences, preferably at least 30 peptide sequences, preferably at least 40 peptide sequences, preferably at least 50 peptide sequences, and so on.

It is to be understood that the peptide sequences of the library generated in step (a) can be of any length, as long as the peptide sequence is capable of being bound by an antibody in the screening process of step (c). In some embodiments, the peptide sequences of the library generated in step (a) comprise, consist or consist essentially of at least 10 amino acids (e.g., 10, 11, 12, 13, 14, 14, 16, 17, 18, 19, 20, 21, 22, 23, 24, 25, 26, 27, 28, 29, 30, 31, 32, 33, 34, 35, 36, 37, 38, 39, 40 or more amino acids) in length. In an embodiment, the library of peptide sequences generated in step (a) comprises peptides of about 15 amino acids to about 50 amino acids. In another embodiment, the library of peptide sequences generated in step (a) comprises peptides of about 20 amino acids in length. In an embodiment, the library of peptide sequences generated in step (a) consists or consists essentially of peptides of about 15 amino acids to about 50 amino acids. In another embodiment, the library of peptide sequences generated in step (a) consists or consists essentially of peptides of about 20 amino acids in length. In some embodiments, the library of peptide sequences generated in step (a) comprises a homogenous population of peptides; that is, where each peptide has the same length. In other embodiments, the library of peptide sequences generated in step (a) comprises a heterogeneous population of peptides; that is, peptides of different length.

In an embodiment, the library of peptide sequences generated in step (a) comprises adjacent and overlapping fragments of the antigen. By “adjacent” is meant that the peptide sequences of the library generated in step (a), when arranged as a continuous amino acid sequence, comprises the amino acid sequence of the antigen in its native reading frame. By “adjacent and overlapping” is meant that a first peptide sequence comprises at least one amino acid residue (e.g., 1, 2, 3, 4, 5, 6, 7, 8, 9, 10, 11, 12, 13, 14, 15, 16, 17, 18, 19, 20 or more amino acid residues) that is in common with a second peptide sequence in the native reading frame of the antigen from which they are derived. For example, where first and second adjacent and overlapping peptide sequences each comprise 20 amino acids, they may comprise amino acid sequences offset by 1, 2, 3, 4 or 5 amino acids, which means they will have 19, 18, 17, 16 and 15 amino acid residues in common, respectively.

In an embodiment, the library of peptide sequences generated in step (a) comprises adjacent fragments that overlap by at least 1 amino acid, preferably by at least 2 amino acids, preferably by at least 3 amino acids, preferably by at least 4 amino acids, preferably by at least 5 amino acids, preferably by at least 6 amino acids, preferably by at least 7 amino acids, preferably by at least 8 amino acids, preferably by at least 9 amino acids, preferably by at least 10 amino acids, preferably by at least 11 amino acids, preferably by at least 12 amino acids, preferably by at least 13 amino acids, preferably by at least 14 amino acids, preferably by at least 15 amino acids, preferably by at least 16 amino acids, preferably by at least 17 amino acids, preferably by at least 19 amino acids and more preferably by at least 19 amino acids. In an embodiment, the library of peptide sequences generated in step (a) comprises adjacent fragments that overlap by at least 2 amino acids. In another embodiment, the library of peptide sequences generated in step (a) comprises adjacent fragments that overlap by at least three amino acids.

In another embodiment, the library of peptide sequences generated in step (a) comprises adjacent and overlapping fragments that are offset by at least 19 amino acid, preferably by at least 18 amino acids, preferably by at least 17 amino acids, preferably by at least 16 amino acids, preferably by at least 15 amino acids, preferably by at least 14 amino acids, preferably by at least 13 amino acids, preferably by at least 12 amino acids, preferably by at least 11 amino acids, preferably by at least 10 amino acids, preferably by at least 9 amino acids, preferably by at least 8 amino acids, preferably by at least 7 amino acids, preferably by at least 6 amino acids, preferably by at least 5 amino acids, preferably by at least 4 amino acids, preferably by at least 3 amino acids, preferably by at least 2 amino acids and more preferably by at least 1 amino acid. In an embodiment, the library of peptide sequences generated in step (a) comprises adjacent and overlapping fragments that are offset by at least 2 amino acids. In another embodiment, the library of peptide sequences generated in step (a) comprises adjacent and overlapping fragments that are offset by at least 3 amino acids.

In some embodiments, the library of peptide sequences generated in step (a), when arranged as a continuous amino acid sequence, comprise the entire amino acid sequence of the native antigen (i.e., the naturally-occurring antigen, as it exists in nature). It will be understood, however, that library of peptide sequences generated in step (a), when arranged as a continuous amino acid sequence, need not comprise the entire sequence of the native antigen, but may comprise instead the extracellular domain of the antigen, or a B cell epitope thereof. Thus, in an embodiment disclosed herein, the library of peptide sequences generated in step (a), when arranged as a continuous amino acid sequence, comprise the amino acid sequence of the extracellular domain of the antigen in its native reading frame. In another embodiment, the library of peptide sequences generated in step (a), when arranged as a continuous amino acid sequence, consists or consists essentially of the amino acid sequence of the extracellular domain of the antigen in its native reading frame.

As used herein, the term “extracellular domain of the antigen” typically refers to a part of the antigen associated with cancer or the checkpoint antigen that is accessible from the outside of said cell, for example, by antibodies with binding specificity for the antigen associated with cancer or the checkpoint antigen. For example, where the antigen associated with cancer or the checkpoint antigen is a transmembrane protein, the extracellular domain will be an extracellular loop or a part thereof or any other extracellular part of the antigen. In some embodiments, the extracellular domain of the antigen comprises at least 5 amino acids (e.g., 6, 7, 8, 9, 10 or more amino acids), preferably at least 8 amino acids, preferably at least 10 amino acids, preferably at least 12 amino acids, preferably at least 15 amino acids, preferably at least 20 amino acids, preferably at least 25 amino acids, preferably at least 30 amino acids, preferably at least 40 amino acids, preferably at least 50 amino acids, preferably at least 60 amino acids or preferably at least 70 amino acids. A transmembrane protein will be understood as meaning an antigen that is anchored or otherwise attached to the plasma membrane of a cell, wherein at least a part of the antigen (comprising the extracellular domain) faces the extracellular space of the cell. A transmembrane protein may be anchored or otherwise attached to the plasma membrane of a cell by one or more transmembrane domains, one or more lipid anchors, or by the interaction with any other protein, lipid, saccharide, or other structure that can be found on the plasma membrane of the cell. For example, the antigen may be a transmembrane protein having an extracellular domain or it may be a protein attached to the surface of a cell through its interacting with another transmembrane protein.

In an embodiment, the library of peptide sequences generated in step (a) comprises a B cell epitope of the antigen associated with cancer or a B cell epitope of the checkpoint antigen. In another embodiment, the library of peptide sequences generated in step (a) consists or consists essentially of a B cell epitope of the antigen associated with cancer or a B cell epitope of the checkpoint antigen. In another embodiment, the library of peptide sequences generated in step (a) comprises at least one B cell epitope of the antigen associated with cancer and at least one B cell epitope of the checkpoint antigen. In another embodiment, the library of peptide sequences generated in step (a) consists or consists essentially of at least one B cell epitope of the antigen associated with cancer and at least one B cell epitope of the checkpoint antigen.

In an embodiment, the library of peptide sequences generated in step (a) comprises adjacent and overlapping fragments of a B cell epitope of the antigen associated with cancer or adjacent and overlapping fragments of a B cell epitope of the checkpoint antigen. In another embodiment, the library of peptide sequences generated in step (a) consists or consists essentially of adjacent and overlapping fragments of a B cell epitope of the antigen associated with cancer or adjacent and overlapping fragments of a B cell epitope of the checkpoint antigen. In another embodiment, the library of peptide sequences generated in step (a) comprises adjacent and overlapping fragments of at least one B cell epitope of the antigen associated with cancer and adjacent and overlapping fragments of at least one B cell epitope of the checkpoint antigen. In another embodiment, the library of peptide sequences generated in step (a) consists or consists essentially of adjacent and overlapping fragments of at least one B cell epitope of the antigen associated with cancer and adjacent and overlapping fragments of at least one B cell epitope of the checkpoint antigen.

In another embodiment, the library of peptide sequences generated in step (a) comprises two or more B cell epitopes of the antigen associated with cancer, or two or more B cell epitopes of the checkpoint antigen. In another embodiment, the library of peptide sequences generated in step (a) consists or consists essentially of two or more B cell epitopes of the antigen associated with cancer, or two or more B cell epitopes of the checkpoint antigen.

In another embodiment, the library of peptide sequences generated in step (a) comprises adjacent and overlapping fragments of two or more B cell epitopes of the antigen associated with cancer, or adjacent and overlapping fragments of two or more B cell epitopes of the checkpoint antigen. In another embodiment, the library of peptide sequences generated in step (a) consists or consists essentially of adjacent and overlapping fragments of two or more B cell epitopes of the antigen associated with cancer, or adjacent and overlapping fragments of two or more B cell epitopes of the checkpoint antigen.

In another embodiment, the library of peptide sequences generated in step (a) comprises two or more B cell epitopes of an extracellular domain of the antigen associated with cancer, or two or more B cell epitopes of an extracellular domain of the checkpoint antigen. In another embodiment, the library of peptide sequences generated in step (a) consists or consists essentially of two or more B cell epitopes of an extracellular domain of the antigen associated with cancer, or two or more B cell epitopes of an extracellular domain of the checkpoint antigen.

In another embodiment, the library of peptide sequences generated in step (a) comprises adjacent and overlapping fragments of two or more B cell epitopes of an extracellular domain of the antigen associated with cancer, or adjacent and overlapping fragments of two or more B cell epitopes of an extracellular domain of the checkpoint antigen. In another embodiment, the library of peptide sequences generated in step (a) consists or consists essentially of adjacent and overlapping fragments of two or more B cell epitopes of an extracellular domain of the antigen associated with cancer, or adjacent and overlapping fragments of two or more B cell epitopes of an extracellular domain of the checkpoint antigen.

In an embodiment, the library of peptide sequences generated in step (a) comprises at least one B cell epitope of the antigen associated with cancer and at least one B cell epitope of the checkpoint antigen.

As used herein, the term “B cell epitope” refers to a part of the antigen (i.e., the antigen associated with cancer or the checkpoint antigen) that is recognized and specifically bound by an antibody. Thus, a “B cell epitope” is to be understood as being a small subsequence of an antigen, said epitope subsequence capable of being recognized by an antibody. An antigen may contain multiple B cell epitopes, and therefore may be bound by multiple distinct antibodies, but any given epitopic fragment of this antigen will typically be bound by only one antibody.

In another embodiment, the library of peptide sequences generated in step (a) comprises at least one mimotope.

As used herein, the term “mimotope” refers to a molecule which has a conformation that has a topology equivalent to the B cell epitope of which it is a mimic and typically binds to the same antigen-binding region of an antibody which binds immunospecifically to said B cell epitope. The mimotope will elicit an immunological response in a host that is reactive to the antigen to which it is a mimic. Methods of producing a vaccine composition comprising a fusion protein that comprises mimotopes, as herein described, have the advantage that the fusion proteins minimize the formation of autoreactive T-cells, since the peptides have an amino acid sequence which varies from those of naturally occurring antigen. Suitable mimotopes for a particular antigen will be familiar to persons skilled in the art.

Mimotopes preferably are antigenic polypeptides which in their amino acid sequence vary from the amino acid sequence of a native B cell epitope of the antigen associated with cancer. The mimotope may not only comprise amino acid substitutions of one or more naturally occurring amino acid residues, but also of one or more non-natural amino acids (i.e. not from the 20 “classical” amino acids) or they may be completely assembled of such non-natural amino acids. Suitable mimotopes may be provided from commercially available peptide libraries. Preferably, these mimotopes are at least 7 amino acids in length (e.g, 7, 8, 9, 10, 11, 12, 13, 14, 15, 16, 17 or more amino acids in length). Preferred lengths may be up to 16, preferably up to 14 or 20 amino acids (e.g. 5 to 16 amino acid residues). Longer mimotopes may also be employed. In another embodiment, the mimotope may be part of a polypeptide and consequently comprising at their N- and/or C-terminus at least one further amino acid residue.

The peptide sequences of the library generated in step (a) can be synthetically produced by chemical synthesis methods which are well known in the art, either as an isolated peptides or as a part of other peptides or polypeptides. Alternatively, the peptide sequences can be produced in a microorganism which produces the (recombinant) peptide sequence or sequences, which can then isolated and, if desired, further purified. The peptide sequences can be produced in microorganisms such as bacteria, yeast or fungi, in eukaryote cells such as a mammalian or an insect cell, or in a recombinant virus vector such as adenovirus, poxvius, herpesvius, Simliki forest virus, baculovirus, bacteriophage, sindbis virus or sendai virus. Suitable bacteria for producing the peptide sequences will be familiar to persons skilled in the art, illustrative examples of which include E. coli, B. subtilis or any other bacterium that is capable of expressing the peptide sequences. Illustrative examples of suitable yeast types for expressing the peptide sequences include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptide sequences are well known in the art and include, for example, gel filtration, affinity chromatography and ion exchange chromatography.

To facilitate isolation of a peptide sequence, a fusion polypeptide may be made wherein the peptide sequence is translationally fused (covalently linked) to a heterologous polypeptide which enables isolation by affinity chromatography. Typical heterologous polypeptides are His-Tag (e.g. His₆. 6 histidine residues), GST-Tag (Glutathione-S-transferase) etc. The fusion polypeptide facilitates not only the purification of the mimotopes but can also prevent the polypeptide from being degraded during purification. If it is desired to remove the heterologous polypeptide after purification the fusion polypeptide may comprise a cleavage site at the junction between the peptide sequence and the heterologous polypeptide. The cleavage site consists of an amino acid sequence that is cleaved with an enzyme specific for the amino acid sequence at the site (e.g. proteases).

In an embodiment, the peptide sequences of the library generated in step (a) are modified at or nearby their N- and/or C-termini so that at said positions a cysteine residue is bound thereto.

In another embodiment, the library of peptide sequences is generated by combinatorial chemistry or by high throughput screening techniques for the most varying structures (see, for example, Display: A Laboratory Manual by Carlos F. Barbas (Editor), et al.; and Willats W G Phage display: practicalities and prospects. Plant Mol. Biol. 2002 December; 50(6):837-54).

In an embodiment disclosed herein, the method of generating the library of peptide sequences in step (a) comprises:
(i) providing two or more nucleic acid sequences encoding the peptide sequences;
(ii) inserting each of the nucleic acid sequences of step (i) into an expression vector to generate a library of expression vectors;
(iii) transforming host cells with each of the expression vectors of step (ii) to generate a population of transformed host cells;
(iv) establishing clonal colonies of the transformed host cells generated in step (iii) to yield individual clones for each expression vector, and
(v) culturing the clonal colonies established in step (iv) under conditions suitable to express the peptide sequences.

In an embodiment disclosed herein, the method of generating the library of peptide sequences in step (a) comprises:
(i) providing one or more DNA molecule(s) each comprising two or more coding elements and a linking element linking two adjacent of said coding elements, wherein the coding elements when arranged as a continuous sequence comprise the entire coding sequence of the gene of the antigen associated with cancer or the checkpoint antigen in its native reading frame and each of said coding elements comprises a DNA sequence which overlaps with the DNA sequence of the adjacent coding element;
(ii) cloning said one or more DNA molecule(s) into a single DNA vector;
(iii) amplifying said DNA vector, transforming a host cell with the DNA vector, propagating transformed host cells, and preparing vector DNA;
(iv) disrupting the DNA vector obtained in step (iii) for releasing the individual DNA molecules encoding the peptide sequences, and amplifying the coding elements by PCR with oligonucleotide primers specific for said linking elements;
(v) cloning each of the individual coding DNA molecules obtained in step (iv) into respective DNA vectors so that each DNA vector comprises an individual coding DNA sequence;
(vi) transforming the DNA vector obtained in step (v) into a host cell and establishing clonal colonies to yield individual clones for each individual coding DNA sequence; and
(vii) culturing said clonal colonies under conditions suitable to express the encoded peptide sequence.

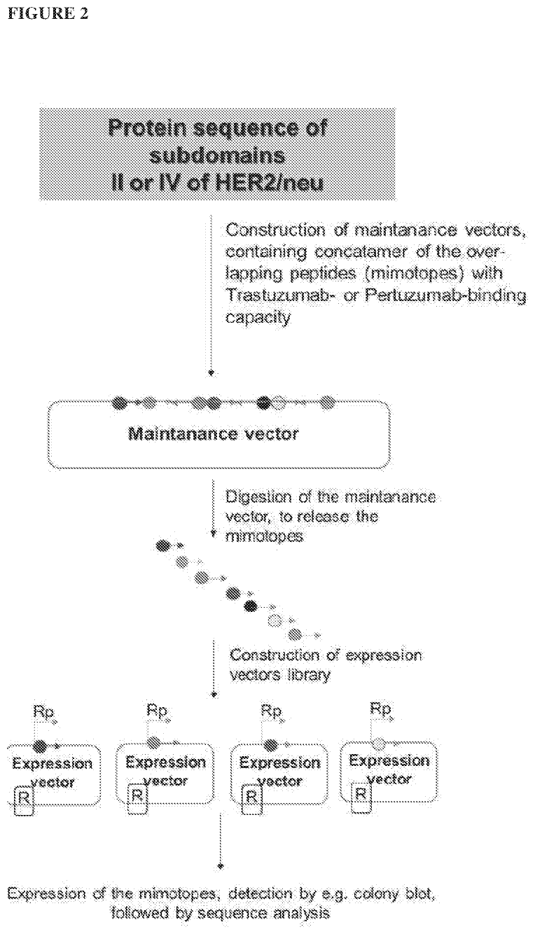

In another embodiment disclosed herein, the library of peptide sequences of step (a) is generated using the PepID technology (ATG:biosynthetics, Germany, GmbH) to generate BioPeptide libraries comprising overlapping peptides sequences, preferably adjacent and overlapping peptides sequences. Applying this technology can increase the likelihood of identifying epitopes with the highest binding capacity to the at least one antibody of step (b). As depicted in FIG. 2, for example, the generated overlapping peptide sequences are first cloned into a maintenance vector which serves as a source vector and can be propagated in E. coli. This means that the overlapping peptide sequences can be regenerated from the maintenance vector and/or the permanent stocks at any time and in almost unlimited quantity, a great advantage which stands out in contrast to random peptides. For generating a library of overlapping peptide sequences, the maintenance vector is digested, allowing the potential to individually obtain each of the over-lapping peptide sequences. Expression vectors containing a gene for a reporter protein (Rp) and one peptide sequence can be constructed, producing an expression library that can be transformed into host cells such as E. coli to yield individual clones for each peptide sequence. Upon induction, each peptide sequence can be expressed in fused form to the reporter gene product. Using immunological assays, such as colony blot (Tobias et al., 2010), the E. coli clones each expressing one peptide sequence can be examined, the expression vector from the positive clones extracted and sequencing applied to identify the sequence of the peptide from the positive E. coli clone.

In other embodiments disclosed herein, one or more of the peptide sequences identified in step (c) may be modified or derivatized to enhance their ability, when administered, to induce an antibody response directed against the antigen associated with cancer or the checkpoint antigen. Suitable modifications and derivations will be familiar to persons skilled in the art, illustrative examples of which include conjugating, coupling or otherwise attaching to the peptide sequence a solubilizing moiety. In other embodiments, functional variants may be include amino acid substitutions and/or other modifications in order to increase the stability of the peptide sequence in the fusion protein and/or to increase the immunogenicity of the peptide sequences. Suitable modifications will be familiar to persons skilled in the art.

### Fusion Protein

As used herein, the term “fusion protein” refers to a non-native peptide composed of two or more peptide sequences linked to one another. In the context of the present disclosure, a fusion protein refers to a non-native peptide composed of two or more of the peptide sequences identified in step (c) to which the antibody of step (b) would bind. Suitable methods of linking peptide sequences will be familiar to persons skilled in the art, illustrative examples of which include peptide (amide) bonds and linkers.

As used herein, the term “linker” refers to a short polypeptide sequence interposed between any two neighboring peptide sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline).

The fusion protein produced by the methods disclosed herein may comprise two or more of the peptide sequences identified in step (c); that is, 2, 3, 4, 5, 6, 7, 8 or more of the peptide sequences identified in step (c). Thus, in an embodiment, the fusion protein comprises at least two of the peptide sequences identified in step (c). In another embodiment, the fusion protein consists or consists essentially of two of the peptide sequences identified in step (c). In another embodiment, the fusion protein comprises at least three of the peptide sequences identified in step (c). In another embodiment, the fusion protein consists or consists essentially of three of the peptide sequences identified in step (c). In another embodiment, the fusion protein comprises at least four of the peptide sequences identified in step (c). In another embodiment, the fusion protein consists or consists essentially of four of the peptide sequences identified in step (c). In another embodiment, the fusion protein comprises at least five of the peptide sequences identified in step (c). In another embodiment, the fusion protein consists or consists essentially of five of the peptide sequences identified in step (c). In another embodiment, the fusion protein comprises at least six of the peptide sequences identified in step (c). In another embodiment, the fusion protein consists or consists essentially of six of the peptide sequences identified in step (c).

In an embodiment disclosed herein, the fusion protein comprises an amino acid sequence selected from the group consisting of SEQ ID NOs:2 to 8.

In another embodiment, the fusion protein comprises, consists, or consists essentially of an amino acid sequence selected from the group consisting of SEQ ID NOs:2, 3 and 4.

As discussed elsewhere herein, the fusion protein produced by the methods disclosed herein may comprise a combination of (i) fragments (e.g., adjacent and overlapping fragments) of one or more B cell epitopes of an antigen associated with cancer and (ii) fragments (e.g., adjacent and overlapping fragments) of one or more B cell epitopes of a checkpoint antigen. Thus, in an embodiment, the fusion protein produced in step (d) of the methods disclosed herein comprises, consists or consists essentially of at least 2, at least 3, at least 4, at least 5, at least 6, at least 7, at least 8, at least 9, at least 10, at least 11 or at least 12 non-contiguous B cell epitopes of the antigen associated with cancer and/or the checkpoint antigen, including fragments thereof (e.g., adjacent and overlapping fragments thereof).

It is to be understood that, where the fusion protein comprises more than one mimotope, the mimotopes may induce an antibody response that raises an antibody against the same antigen (i.e., the antigen associated with cancer or the checkpoint antigen), or they may each raise an antibody response against different antigens (i.e., the antigen associated with cancer or the checkpoint antigen). In an embodiment disclosed herein, the method disclosed herein produces a fusion protein that comprises, consists or consists essentially of at least one, at least 2, at least 3, at least 4, at least 5 or at least 10 mimotopes.

Also contemplated herein are methods in which the fusion protein that is produced in step (d) comprises the at least two peptide sequences, as herein described, concatenated two or more times in tandem repeat. For example, fusion proteins produced in step (d) may comprise two or more tandem repeats of a B cell epitope (or a mimotope of a B cell epitope) of an antigen associated with cancer and/or a checkpoint antigen, including fragments thereof, whether the fragments are adjacent and overlapping or not. It will be understood that incorporating two or more different B cell epitopes (or a mimotope of a B cell epitope) of an antigen associated with cancer and/or a checkpoint antigen, including fragments thereof, whether the fragments are adjacent and overlapping or not, is more likely to generate a more beneficial immune response by eliciting antibodies that specifically recognize (bind to) multiple B cell epitopes of the native antigen.

Suitable methods of combining the at least two peptide sequences identified in step (c) to produce a fusion protein according to step (d), as herein described, would be familiar to persons skilled in the art. An illustrative example includes peptide synthesis that involves the sequential formation of peptide bonds linking each peptide sequence, as herein described, to its respectively neighboring peptide sequence, and recovering said fusion peptide. Illustrative examples include the methods described in “Amino Acid and Peptide Synthesis” (Oxford Chemistry Primers; by John Jones, Oxford University Press). Synthetic peptides can also be made by liquid-phase synthesis or solid-phase peptide synthesis (SPPS) on different solid supports (e.g. polystyrene, polyamide, or PEG). SPPS may incorporate the use of F-moc (9H-fluoren-9-ylmethoxycarbonyl) or t-Boc (tert-Butoxycarbonyl). Custom peptides are also available from a number of commercial manufacturers.

Alternatively, the fusion protein may be produced in accordance with step(d) by recombinant methodology. For example, a nucleic acid molecule comprising a nucleic acid sequence encoding the fusion protein can be transfecting into a suitable host cell capable of expressing said nucleic acid sequence, incubating said host cell under conditions suitable for the expression of said nucleic acid sequence, and recovering said fusion protein. Suitable methods for preparing a nucleic acid molecule encoding the fusion protein will also be known to persons skilled in the art, based on knowledge of the genetic code, possibly including optimizing codons based on the nature of the host cell (e.g. microorganism) to be used for expressing and/or secreting the recombinant fusion protein. Suitable host cells will also be known to persons skilled in the art, illustrative examples of which include prokaryotic cells (e.g., E. coli) and eukaryotic cells (e.g., P. pastoris). Reference is made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated, typically in a host cell, to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product. In some embodiments, the nucleic acid sequence encoding the peptide sequences, as herein described, or the fusion proteins, as herein described, are codon-optimised for expression in a suitable host cell. For example, where the fusion protein is to be used for inducing a humoral response against HER-1 in a human subject, the nucleic acid sequences can be human codon-optimised. Suitable methods for codon optimisation would be known to persons skilled in the art, such as using the “Reverse Translation” option of “Gene Design” tool located in “Software Tools” on the John Hopkins University Build a Genome website.

As noted elsewhere herein, the at least two peptide sequences that are combined in step (d) to produce the fusion protein, as herein described, can be linked to one another within the fusion peptide by any means known to persons skilled in the art. The terms “link” and “linked” include direct linkage of two peptide sequences via a peptide bond; that is, the C-terminus of one peptide sequence is covalently bound via a peptide bond to the N-terminal of another peptide sequence. The terms “link” and “linked” also include within their meaning the linkage of two peptide sequences via an interposed linker element.

In an embodiment disclosed herein, the at least two peptide sequences are linked to one another via a non-native linker peptide sequence.

Where the at least two peptide sequences comprise amino acid sequences found in nature (e.g., sequences derived from a B cell epitope of the native antigen), the peptide sequences are linked to one another within the fusion peptide in such a way as to ensure the fusion protein comprises an amino acid sequence that is not identical to a continuous stretch of at least 50 amino acid residues of the native antigen associated with cancer or the native checkpoint antigen. Thus, in an embodiment disclosed herein, the order in which the peptide sequences are combined in step (d) does not result in a fusion protein comprising an amino acid sequence that is identical to a continuous stretch of at least 50 amino acid residues of the antigen associated with cancer or the checkpoint antigen. In another embodiment, where the at least two peptide sequences are B cell epitopes of the native antigen associated with cancer or the native checkpoint antigen, the fusion protein comprises a least two non-contiguous B cell epitopes of the antigen associated with cancer or the checkpoint antigen; that is, linked to one another such that the at least two B cell epitopes are non-contiguous in their native state; that is, in the native antigen associated with cancer or the native checkpoint antigen.

By linking the at least two peptide sequences identified in step (c) to produce a fusion protein in accordance with step (d), a homogeneous formulation can be achieved in which only one kind of fusion protein is present. The elements of the fusion protein (i.e. the peptide sequences capable of inducing antibody responses in which the antibodies bind to B cell epitopes of the native antigen associated with cancer and/or the native checkpoint antigen) are the same in every fusion protein and can be chosen (or chosen and modified) such that undesired intra- and inter-polypeptide interactions are minimized.

As described elsewhere herein, the fusion protein produced in accordance with step (d) of the methods disclosed herein comprises at least two peptide sequences (e.g., 2, 3, 4, 5, 6, 7, 8, 9, 10, 11 or more peptide sequences), wherein each of the at least two peptide sequences induces an antibody response in which the antibody binds to a B cell epitope of the native antigen associated with cancer and/or the native checkpoint antigen.

In some embodiments, the peptide sequences of the library generated in step (a) comprise functional variants of fragments of the antigen associated with cancer or the checkpoint antigen. As used in this context, a “functional variant” will typically comprise an amino acid sequence that differs from the amino corresponding amino acid sequence of the native antigen (i.e., the antigen associated with cancer or the checkpoint antigen) by one or more (e.g., 1, 2, 3, 4, 5, 6, 7, 8, 9 or more) amino acid substitutions, wherein said difference (i) does not, or does not completely, abolish the capacity of the variant to bind to an antibody of step (b) that would otherwise bind to the native sequence and (ii) does not, or does not completely, abolish the capacity of the variant, when administered within the fusion protein, to induce an antibody response in which the antibody binds to a B cell epitope of native antigen associated with cancer or the native checkpoint antigen.

In some embodiments, the functional variant may comprise amino acid substitutions that enhance the capacity of the peptide sequence to induce an antibody response in which the antibody binds to a B cell epitope of native antigen associated with cancer or the native checkpoint antigen, as compared to the native sequence of that B cell epitope. In an embodiment, the functional variant differs from the native peptide sequence by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, the functional variant has at least 85% sequence identity to the amino acid sequence of a fragment of the native antigen associated with cancer or the native checkpoint antigen. Reference to “at least 85%” includes 85%, 86%, 87%, 88%, 89%, 90%, 91%, 92%, 93%, 94%, 95%, 96%, 97%, 98%, 99% or 100% sequence identity or similarity, for example, after optimal alignment or best fit analysis. Thus, in an embodiment, the sequence has at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity or sequence homology to the amino acid sequence of a fragment of the native antigen associated with cancer or the native checkpoint antigen, for example, after optimal alignment or best fit analysis.

The terms “identity”, “similarity”, “sequence identity”, “sequence similarity”, “homology”, “sequence homology” and the like, as used herein, mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. In an embodiment, the amino acid sequences may be modified by way of conservative substitution of any of the amino acid residues contained therein, such that the modification has no effect on the binding specificity or functional activity of the modified polypeptide when compared to the unmodified polypeptide.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

In an embodiment disclosed herein, the fusion protein further comprises a T cell epitope of an antigen associated with cancer. Illustrative examples of suitable T cell epitopes include T cell epitopes of EGFR (e.g., Her2/neu, Her-1), WT1 (Wilms Tumor antigen), BAGE (B melanoma antigen), CEA (carcinoembryonic antigen), CpG (cytosine-phosphate diesterguanine), Gp100 (glycoprotein 100), h-TERT (telomerase transcriptase), MAGE (melanoma antigen-encoding gene), Melan-A (melanoma antigen recognized by T cells), NY-ESO-1 and MUC-1 (mucin-1). In an embodiment, the T cell epitope is a cytotoxic T cell epitope.

Antibodies that Bind to an Antigen Associated with Cancer or a Checkpoint Antigen

As described elsewhere herein, step (b) comprises obtaining at least one antibody that bind to the antigen associated with cancer or the checkpoint antigen and step (c) comprises screening the library of peptide sequences generated in step (a) to identify at least two peptide sequences that specifically bind to the at least one antibody. In some embodiments, the at least one antibody is a polyclonal antibody, which may be prepared by immunizing a subject with the antigen (which may comprise multiple doses of the antigen). The polyclonal antibody may first be isolated from sera from the immunized subject subsequent to the immunization schedule before performing the screening process of step (c). Alternatively, step (c) can be performed using the sera collected from the immunized subject. In an embodiment, the at least one antibody is a monoclonal antibody. In another embodiment, the at least one antibody is a therapeutic antibody.

In other embodiments disclosed herein, step (b) comprises screening the library of peptide sequences generated in step (a) with the two or more antibodies to identify at least two peptide sequences that specifically bind to the two or more antibodies. In another embodiment disclosed herein, step (b) comprises screening the library of peptide sequences generated in step (a) with the two or more antibodies, wherein each of the two or more antibodies specifically binds to a different peptide sequence in the library generated in step (a). By two or more antibodies means 2, 3, 4, 5, 6, 7, 8, 9, 10, 11 or more antibodies. Thus, in an embodiment, step (b) comprises screening the library of peptide sequences generated in step (a) with at least 2 antibodies, preferably at least 3 antibodies, preferably at least 4 antibodies, preferably at least 5 antibodies, preferably at least 6 antibodies, preferably at least 7 antibodies or more preferably at least 8 antibodies. In another embodiment, step (b) comprises screening the library of peptide sequences generated in step (a) with at least 2 antibodies, preferably at least 3 antibodies, preferably at least 4 antibodies, preferably at least 5 antibodies, preferably at least 6 antibodies, preferably at least 7 antibodies or more preferably at least 8 antibodies, wherein each of the antibodies binds specifically to a different peptide sequence in the library generated in step (a).

In an embodiment disclosed herein, the two or more antibodies comprises a monoclonal antibody.

In an embodiment disclosed herein, the two or more antibodies comprises a therapeutic antibody. Suitable therapeutic antibodies (i.e., which are known to bind to the native antigen in vivo) will be familiar to persons skilled in the art. Illustrative examples of therapeutic antibodies that bind to an antigen associated with cancer include anti-EGFR antibodies such as trastuzumab, pertuzumab, cetuximab, panitumumab, zalutumumab, nimotuzumab, matuzumab, necitumumab, and anti-checkpoint antigen antibodies such as nivolumab, ipilimumab, pembrolizumab, aveumab and atezolizumab.

Antibodies suitable for use in accordance with the methods disclosed herein also include humanized, single-chain, chimeric, synthetic, recombinant, hybrid, mutated and CDR-grafted antibodies. Antibodies may be derived from any species and may be of any suitable isotype, such as IgG, IgM, IgA, IgD, IgE or any subclass thereof. Persons skilled in the art will appreciate that antibodies produced recombinantly, or by other means, for use in accordance with the present invention include antigen-binding fragments thereof that can still bind to or otherwise recognize the primary binding agent. Illustrative examples of suitable antigen-binding fragments of antibodies include Fab, an F(ab)₂, Fv and scFv fragments.

### Screening

Methods of screening the library generated in step (a) with an antibody to identify peptide sequences within the library that specifically bind to the antibody will be familiar to persons skilled in the art, an illustrative example of which is described elsewhere herein.

A suitable method is a colony blot assay, as described in Tobias et al., 2010. For example, a portion of the library of peptide sequences is plated onto selective plates to obtain single colonies. The obtained colonies are streaked onto duplicate LB agar plates. One plate can be used for the colony blot assay, using nitrocellulose membrane, which will be treated according to standard methods for lysis of the cells and preparation for detection of clones binding to one or more antibodies. The detection of binding can be carried out using an appropriate secondary antibody conjugated to detectable label (e.g., an enzyme)) allowing detection of the colonies to which an antibody has bound. Positive colonies can then be picked from the matched duplicate plate, grown and plasmids will be prepared. The sequences of the inserts in the individual plasmids can then be determined by sequencing using methods known in the art.

Other illustrative examples of suitable detectable labels that can be used include fluorophores, radioactive isotopes, chromophores, electrochemiluminescent labels, bioluminescent labels, polymers, polymer particles, beads or other solid surfaces, gold or other metal particles or heavy atoms, spin labels, haptens, myc, nitrotyrosine, biotin and avidin. Others include phosphor particles, doped particles, nanocrystals or quantum dots.

In an embodiment disclosed herein, a direct detectable label is used. Direct detectable labels may be detected per se without the need for additional molecules. In another embodiment, an indirect detectable label is used, which requires the employment of one or more additional molecules so as to a form detectable molecular complex (e.g., a biotin-avidin complex).

The terms “recognize”, “recognizing”, “bind”, “binding” and the like, as used herein, mean an event in which an antibody (including an antigen-binding fragment thereof), directly or indirectly interacts with a target antigen (peptide sequence) in such a way that the interaction with the target may be detected. The terms “specific for”, “specifically” and the like, as used herein to describe binding of an antibody (or an antigen-binding fragment thereof) through a specific interaction between complementary binding partners, rather than through non-specific aggregation or association.

### Carrier

In an embodiment disclosed herein, the method further comprises attaching the fusion protein to a carrier.

It will be understood by persons skilled in the art that, where coupling of the a fusion protein to the carrier protein is via a linker, it is preferable to effect such linker-mediated coupling from the C-terminus of the fusion protein, since linker coupling from the N-terminus may, in some instances, have a negative influence on the desired immune response to be elicited.

In an embodiment disclosed herein, the carrier is immunogenic.

In an embodiment disclosed herein, the carrier is selected from the group consisting of keyhole limpet hemocyanin (KLH), tetanus toxoid (TT), B subunit of cholera toxin (CT, CTB), heat labile toxin (LT) of E. coli and mutants thereof, lactic acid bacteria (LAB), bacterial ghost, liposome, chitosome, virosome, dendritic cell and diphtheria toxin variant CRM-197 (e.g., GenBank Accession No. 1007216A or SEQ ID NO:1).

In an embodiment disclosed herein, the carrier is diphtheria toxin variant CRM-197. CRM-197 (GenBank Accession No. 1007216A) is an enzymatically inactive and nontoxic form of diphtheria toxin that contains a single amino acid substitution (Gly-Glu) at amino acid residue 52. A single GCA mutation that leads to the Glu52 substitution distinguishes CRM-197 from its wild-type species. The absence of toxicity of CRM-197 appears to be due to the loss of enzymatic activity of its fragment A, which in the wild-type species catalyzes the chemical modification of elongation factor 2 (translocase) in infected cells that is essential for protein synthesis. This non-toxic property makes CRM-197 a suitable carrier protein for the preparation of conjugated vaccines.

(CRM-197; GenBank Accession No. 1007216A)
SEQ ID NO: 1
GADDVVDSSKSFVMENFSSYHGTKPGYVDSIQKGIQKPKSGTQGNYDDD
WKEFYSTDNKYDAAGYSVDNENPLSGKAGGVVKVTYPGLTKVLALKVDN
AETIKKELGLSLTEPLMEQVGTEEFIKRFGDGASRVVLSLPFAEGSSSV
EYINNWEQAKALSVELEINFETRGKRGQDAMYEYMAQACAGNRVRRSVG
SSLSCINLDWDVIRDKTKTKIESLKEHGPIKNKMSESPNKTVSEEKAKQ
YLEEFHQTALEHPELSELKTVTGTNPVFAGANYAAWAVNVAQVIDSETA
DNLEKTTAALSILPGIGSVMGIADGAVHHNTEEIVAQSIALSSLMVAQA
IPLVGELVDIGFAAYNFVESIINLFQVVHNSYNRPAYSPGHKTQPFLHD
GYAVSWNTVEDSIRRTGFQGESGHDIKITAENTPLPIAGVLLPTIPGKL
DVNKSKTHISVNGRKIRMRCRAIDGDV

Methods by which a fusion protein can be conjugated, coupled or otherwise attached to a carrier (e.g., CRM-197) are known to persons skilled in the art. Illustrative examples include those described by Chang et al. (1998, FEBS Letters, 427:362-366) and Berti et al. (2004, Biophysical Journal, 86:3-9).

Conjugation of a fusion protein, as herein described, to a carrier is typically achieved through activation of the lysyl residues using suitable crosslinkers. For instance, since CRM-197 contains 40 lysines residues, and many of them are available for crosslinking, the end products of CRM-197 conjugation are invariably heterogeneous. Without being bound by theory or a particular mod of action, it is generally understood that the ratio of fusion protein to carrier protein depends on the size or molecular weight of the fusion protein. For instance, where the fusion protein is relatively small (e.g., about 75 amino acids in length), it may be possible to produce a carrier that is conjugated with 20-39 fusion proteins. Conversely, for a larger fusion protein, the carrier may be conjugated with up to, or fewer than, 20 fusion proteins. In an embodiment described herein, the carrier comprises from 2 to 39 fusion proteins. In another embodiment, the carrier comprises at least 20 fusion proteins. In yet another embodiment, the carrier comprises from 6 to 12 fusion proteins.

The fusion protein can be coupled to the carrier by a covalent bond. However, in some embodiments, the fusion protein may be coupled to the carrier by a non-covalent association. Where the fusion protein is non-covalently associated with the carrier, the non-covalent association will typically involve an electromagnetic interaction between one or more atoms of the fusion protein with one or more atoms of the carrier. Illustrative examples include ionic bonding (i.e., the attraction formed between two oppositely charged ions by virtue of this opposite charge), Van der Weals forces (i.e., forces between permanent and/or induced dipoles of existing covalent bonds within the fusion protein and the carrier) and/or hydrophobic interactions (i.e., forces resulting from the tendency of hydrophobic/aliphatic portions within the fusion protein(s), as herein described, to associate with hydrophobic portions of the carrier).

In an embodiment disclosed herein, the carrier comprises from 2 to 39 fusion proteins. In an embodiment disclosed herein, the carrier comprises from 6 to 12 fusion proteins.

### Adjuvant

In an embodiment disclosed herein, the vaccine composition produced by the methods disclosed herein further comprises an adjuvant.

As used herein, the term “adjuvant” typically refers to a class of substance that can increase the magnitude of the immune response elicited by the fusion protein beyond that which would be expected, either from the fusion protein alone or from the fusion peptide-carrier conjugate, as herein described, in the absence of an adjuvant.

Suitable adjuvants will be known to persons skilled in the art. Non-limiting examples of suitable adjuvants include aluminium salts (e.g. aluminium hydroxide, aluminium phosphate and potassium aluminium sulfate (also referred to as Alum)), liposomes, virosomes, water-in-oil or oil-in-water emulsions (e.g. Freund's adjuvant, Montanide®, MF59@ and AS03), 3-O-desacyl-4′-monophosphoryl lipid A (MPL) and adjuvants containing MPL (e.g. AS01, AS02 and AS04) and saponin-based adjuvants. Saponin-based adjuvants include saponins or saponin derivatives from, for example, Quillaja saponaria, Panax ginseng Panax notoginseng, Panax quinquefolium, Platycodon grandiflorum, Polygala senega, Polygala tenuifolia, Quillaja brasiliensis, Astragalus membranaceus and Achyranthes bidentata. Exemplary saponin-based adjuvants include iscoms, iscom matrix, ISCOMATRIX™ adjuvant, Matrix M™ adjuvant, Matrix C™ adjuvant, Matrix Q™ adjuvant, AbISCO@-100 adjuvant, AbISCO@-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative. The vaccine composition as herein described can also be associated with immumodulatory agents, including, for example, cytokines, chemokines and growth factors. Mixtures of two or more adjuvants within the same vaccine composition are also contemplated herein.

In an embodiment disclosed herein, the adjuvant is selected from the group consisting of aluminium hydroxide, aluminium phosphate, potassium aluminium sulfate, calcium phosphate hydroxide, a saponin, Freund's complete adjuvant, a TLR agonist, CRM197, Montanide®, Freund's incomplete adjuvant, MF59, a CpG oligonucleotide, iscoms, iscom matrix, ISCOMATRIX™ adjuvant, Matrix M™ adjuvant, Matrix C™ adjuvant, Matrix Q™ adjuvant, AbISCO®-100 adjuvant, AbISCO®-300 adjuvant, ISCOPREP™, an ISCOPREP™ derivative, adjuvant containing ISCOPREP™ or an ISCOPREP™ derivative, monophosphoryl lipid A ((3-O-desacyl-4′-monophosphoryl lipid A, MPL), AS01 (a liposome-based formulation of MPL and QS-21), AS04 (a liposome-based formulation of MPL and aluminium hydroxide), QS-21, a QS-21 derivative, an adjuvant containing QS-21 or a QS21 derivative, and a combination of any of the foregoing.

In an embodiment disclosed herein, the adjuvant is a TLR-4 agonist. In an embodiment disclosed herein, the TLR-4 agonist is monophosphoryl lipid A. In an embodiment disclosed herein, the adjuvant is Montanide. In an embodiment disclosed herein, the TLR-4 agonist is AS01 (a liposome-based formulation of MPL and QS-21). In an embodiment disclosed herein, the adjuvant is ASO4 (a liposome-based formulation of MPL and aluminium hydroxide). In an embodiment disclosed herein, the adjuvant is aluminium hydroxide or aluminium phosphate.

The present disclosure also extends to the use of a combination of adjuvants and/or carriers.

### Checkpoint Inhibitors

An emerging approach to immunotherapy involves the use of antibodies that inhibit checkpoint antigens (see, e.g., Pardoll, 2012, Nature Reviews Cancer 12:252-64). As noted elsewhere herein, checkpoints antigens are endogenous inhibitory pathways for immune system function that act to maintain self-tolerance and modulate the duration and extent of immune response to antigenic stimulation. Checkpoint antigens include CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3) and several others, as discussed, for example, in Pardoll (2012, Nature Reviews Cancer 12:252-64) and Nirschl & Drake 2013 Clin Cancer Res 19:4917-24). Antibodies against several of these checkpoint antigens (e.g., CTLA4, PD1, PD-L1) are in clinical trials and have shown unexpected efficacy against tumours that were otherwise resistant to standard therapy. Thus, in an embodiment disclosed herein, and with a view to improving the efficacy of the vaccine composition produced by the methods disclosed herein in the treatment of a cancer, the vaccine composition further comprises a checkpoint inhibitor.

The term “checkpoint inhibitor” will be understood by persons skilled in the art as meaning molecules that inhibit, reduce or otherwise interfere with or modulate one or more checkpoint antigens, either totally or partially. Illustrative examples of suitable checkpoint inhibitors include antibodies and antigen-binding fragments thereof (e.g., Fab fragments) to checkpoint proteins. Suitable checkpoint proteins will be known to persons skilled in the art, illustrative examples of which include CTLA-4 and its ligands CD80 and CD86; PD1 and its ligands PDL1 and PDL2; OX40 and its ligand OX40L; LAG-3 and its ligand MHC class I or II; TIM-3 and its ligand GAL-9; and B- and T-lymphocyte attenuator (BTLA) and its ligand herpes virus entry mediator (HVEM).

In an embodiment disclosed herein, the vaccine composition further comprises a checkpoint inhibitor such that the fusion protein and the checkpoint inhibitor are present in the same composition. It will be understood, however, that the ability of the checkpoint inhibitor to improve the efficacy of the fusion protein disclosed herein does not require the checkpoint inhibitor to be present in the vaccine composition with the fusion protein or to be administered simultaneously to a subject in need thereof. Thus, in some embodiments, the checkpoint inhibitor is administered subsequent to the administration of the vaccine composition, wherein the period of time between administering the vaccine composition and administering the checkpoint inhibitor can be optimised to provide the desired synergistic or additive effect. In a preferred embodiment, the checkpoint inhibitor is administered after the vaccine composition has had enough time to induce an antibody response in the subject to which it is administered. For example, the checkpoint inhibitor is administered after at least 4 days (e.g., 4 days, 5 days, 6 days, 7 days, 8 days, 9 days, 10 days, 11 days, 12 days, 13 days or at least 14 days) after the administration of the vaccine composition or after the last dose of the vaccine composition where a multiple immunization regimen is adopted. The time course can readily be determined by persons skilled in the art.

In another aspect of the present disclosure, there is provided a vaccine composition produced by the methods disclosed herein.

### Pharmaceutical Composition

In another aspect of the present disclosure, there is provided a pharmaceutical composition comprising (i) the vaccine composition produced by the methods disclosed herein and (ii) a pharmaceutically acceptable excipient.

Suitable pharmaceutically acceptable excipients (e.g. carriers, diluents, etc.). will be known to persons skilled in the art. For example, a variety of aqueous (pharmaceutically acceptable) excipients may be used, such as buffered water, 0.4% saline, 0.3% glycine, hyaluronic acid and the like. These compositions may be sterilized by conventional, well known sterilization techniques or may be sterile-filtered. The resulting aqueous solutions may be packaged for use as is or lyophilized, the lyophilized preparation being combined with a sterile solution prior to administration. The compositions may further comprise pharmaceutically acceptable auxiliary substances as required to approximate physiological conditions, such as pH-adjusting and buffering agents, tonicity-adjusting agents, wetting agents and the like, for example sodium acetate, sodium lactate, sodium chloride, potassium chloride, calcium chloride, sorbitan monolaurate, triethanolamine oleate, sucrose or other carbohydrates, among many others. Suitable methods for preparing parenterally administrable compounds will be known or apparent to those skilled in the art and are described in more detail in, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel el al., eds 7.sup.th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutical composition may be in a form suitable for parenteral administration (e.g., subcutaneous, intramuscular or intravenous injection) or in an aerosol form suitable for administration by inhalation, such as by intranasal inhalation or oral inhalation.

The pharmaceutical compositions described herein may also be provided in a kit.

The kit may comprise additional components to assist in performing the methods as herein described, such as administration device(s), excipients(s), carrier(s) and/or diluent(s). The kits may include containers for housing the various components and instructions for using the kit components in such methods.

In an embodiment disclosed herein, the pharmaceutical composition comprises a checkpoint inhibitor, as herein described.

### Uses and Methods for Treating Cancer

In another aspect of the present disclosure, there is provided a method of treating a cancer characterised by an overexpression of an antigen associated with the cancer, the method comprising administering to a subject in need thereof the vaccine composition produced by the methods disclosed herein or the pharmaceutical composition as herein described.

The present disclosure also extends to use of the vaccine composition produced by the methods disclosed herein in the manufacture of a medicament for treating a cancer characterized by an overexpression of an antigen associated with the cancer in a subject in need thereof.

The present disclosure also extends to the vaccine composition produced by the methods disclosed herein, or the pharmaceutical composition as herein described, for use in treating a cancer characterized by an overexpression of an antigen associated with the cancer in a subject in need thereof.

Illustrative examples of cancers that may be the target of treatment by such methods are described elsewhere herein and include breast cancer, ovarian cancer, endometrial cancer, gastric cancer, pancreatic cancer, prostate cancer and salivary gland cancer. In an embodiment, the cancer is breast cancer. In another embodiment, the cancer is gastric cancer.

The vaccine or pharmaceutical compositions, as described herein, are typically administered in an “effective amount”; that is, an amount effective to elicit any one or more inter alia of a therapeutic effect. Persons skilled in the art would be able, by routine experimentation, to determine an effective, non-toxic amount to administer for the desired outcome. In general, the vaccine and/or pharmaceutical compositions, as disclosed herein, can be administered in a manner compatible with the route of administration and physical characteristics of the recipient (including health status) and in such a way that it elicits the desired effect(s) (i.e. therapeutic effect). For example, the appropriate dosage of a composition may depend on a variety of factors including, but not limited to, a subject's physical characteristics (e.g., age, weight, sex), whether the composition is being used as single agent or as part of adjunct therapy (e.g., with a checkpoint antigen or checkpoint inhibitor), the progression (i.e., pathological state) of any underlying cancer, and other factors that may be recognized by persons skilled in the art. Other illustrative examples of general considerations that may be considered when determining, for example, an appropriate dosage of the compositions are discussed by Gennaro (2000, “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press).

It is expected that the effective amount will fall in a relatively broad range that can be determined through methods known to persons skilled in the art, having regard to some of the considerations outlined above.

An effective amount of the fusion protein to be administered will generally be in a range of from about 5 μg to about 1.0 mg of fusion protein per subject, from about 10 μg to about 500 μg of fusion protein per subject, or from about 15 μg to about 60 μg of fusion protein per subject. An effective amount can be ascertained, for example, by standard methods involving measurement of antigen-specific antibody titres. The level of immunity provided by the compositions herein described can be monitored to determine the need, if any, for boosters. For instance, following an assessment of an antigen-specific antibody titre in the serum, typically days or weeks following the first administration of the composition in a subject, optional booster immunisations may be required and/or desired.

The vaccine and/or pharmaceutical compositions, as described herein, can be administered to a subject in need thereof in isolation or in combination with additional therapeutic agent(s); that is, as part of an adjunct therapy. In the context of adjunct therapy, the administration may be simultaneous or sequential; that is, the vaccine and/or pharmaceutical composition is administered first, followed by administration of the additional therapeutic and/or prophylactic agent(s), or the vaccine and/or pharmaceutical composition is administered following the administration of the additional therapeutic agent(s). Thus, where two or more entities are administered to a subject “in conjunction”, they may be administered in a single composition at the same time, or in separate compositions at the same time, or in separate compositions separated in time.

The additional therapeutic agent(s) may comprise a checkpoint inhibitor, as described elsewhere herein. Thus, in an embodiment, the methods disclosed herein further comprise administering to the subject an effective amount of a checkpoint inhibitor, as herein described.

It will be apparent to persons skilled in the art that the optimal quantity and spacing of individual dosages, if required to induce the desired immune response, can be determined, for example, by the form, route and site of administration, and the nature of the particular subject to be treated, as is described elsewhere herein. Optimum conditions can be determined using conventional techniques known to persons skilled in the art.

In some instances, it may be desirable to have several or multiple administrations of the vaccine and/or pharmaceutical compositions, as herein described. For example, the compositions may be administered 1, 2, 3, 4, 5, 6, 7, 8, 9, 10, or more times. The administrations may be from about one day intervals to about twelve week intervals, and in certain embodiments from about one to about four week intervals. Periodic re-administration may be required to achieve a desirable therapeutic result, such as a reduction in tumour size and/or a reduction in the occurrence of metastases. It will also be apparent to persons skilled in the art that the optimal course of administration can be ascertained using conventional course of treatment or efficacy or immune status determination tests.

It will be understood that “inducing” an immune or antigen-specific antibody response, as contemplated herein, includes eliciting or stimulating an immune response and/or enhancing a previously existing immune response to obtaining a desired therapeutic effect, such as a reduction in tumour size, a slowing of tumour growth and/or a reduction in the occurrence of metastases. The effect can also be prophylactic in terms of, for example, completely or partially preventing the occurrence of metastases.

As used herein, the terms “administration” or “administering” typically refer to the step of introducing the vaccine and/or pharmaceutical compositions, as herein described, into a patient's body so that the patient's immune system mounts a response to the peptide sequences within the fusion protein. As used herein, a “subject in need thereof” includes an individual who has been diagnosed with cancer, wherein the cancer cells express or overexpress an antigen associated with the cancer, as herein described. In its broadest sense, the term “a patient in need thereof” therefore encompasses individuals with an already present need, as well as individuals who are at risk of developing cancer.

As used herein, a medicament which “treats” cancer will ideally eliminate the disease altogether by eliminating its underlying cause so that, upon cessation of administration of the composition, the disease does not re-develop, but remains in remission. As used herein, a medicament which “ameliorates” cancer does not eliminate the underlying cause of the disease, but reduces the severity of the disease as measured by any established grading system and/or as measured by an improvement in the patient's well-being, e.g. decrease in pain and/or discomfort.

All patents, patent applications and publications mentioned herein are hereby incorporated by reference in their entireties.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

These examples set out to generate an anti-cancer vaccine targeting HER-2/neu.

### Background

The proto-oncogene HER-2/neu, also known as ErbB-2, encodes a 185 kDa transmembrane receptor protein and is a member of the erbB/epidermal growth factor receptor (EGFR)/class I family of receptor tyrosine kinases. Approximately 20% of breast cancers exhibit HER-2 gene amplification/overexpression (HER-2/neu positivity), resulting in an aggressive tumor phenotype and reduced survival (Salmon et al., 1987; Salmon et al., 1989). Also, 10% to 25% of gastric cancers (stomach or gastro-oesophageal junction) show either HER-2/neu overexpression or gene amplification and, as in breast cancer, this alteration is associated with an adverse prognosis (Slamon et al., 1987; Yonemura et al., 1991). Due to the involvement of HER-2/neu in tumor progression, its elevated expression in (HER-2/neu overexpressing) tumor cells, and the presence of endogenous HER-2/neu-specific antibodies (Abs) and T cell immune responses in cancer patients (Disis el al., 1997; Ward et al., 1999 Pupa et al., 2004), this oncoprotein represents an excellent target for the development of therapeutic agents.

All the receptors in the EGFR/class I family, which also comprises of HER-1, HER-3 and HER-4, are single-chain membrane-spanning proteins with significant sequence homology to one another, with extracellular, transmembrane and intracellular domain regions (Figure-1; Schlessinger and Ullrich, 1992). Receptor-specific ligands have only been identified for HER-1, HER-3 and HER-4. Upon specific ligand-receptor binding, an extended configuration is promoted resulting in hetero-dimerization of these receptors to HER-2/neu. HER-2/neu, however, is constitutively in an extended configuration leading to preferential hetero-dimerization between HER-2/neu2 and other receptors in the family, or homo-dimerization which can presumably be driven by substantial over-expression of HER-2/neu2. These interactions result in receptor activation, mitogenic signaling and cellular proliferation and survival (Yarden and Sliwkowski, 2001; Burgess et al., 2003).

### Anti-HER-2/Neu Immunotherapy

Current treatment of cancer and in particular of breast cancer has been impressively diversified since the introduction of monoclonal antibodies (mAb) such as the anti-HER-2/neu mAb Trastuzumab. This mAb, which binds to the subdomain IV of the HER-2/neu receptor (Figure-1), was introduced in the late 1990's and is approved by the U.S. Food and Drug Administration (FDA) for passive immunotherapy in the treatment of patients with advanced HER-2/neu overexpressing breast cancer. Twenty six percent (26%) of women with HER-2-positive metastatic breast cancer (MBC) respond to Trastuzumab as a single agent (Vogel et al., 2002). It was shown that the response rate to Trastuzumab in combination with chemotherapy is increased significantly compared with chemotherapy alone (Eirmann, 2001, Marthy et al., 2005). The clinical use of Trastuzumab has resulted not only in significant prolongation of disease-free and overall survival in early breast cancer, but also in the significant prolongation of overall survival in patients with MBC overexpressing the HER-2/neu protein (Slamon et al., 2001). In a recent study, it was shown that women with HER-2/neu-positive disease who received Trastuzumab had improved prognosis compared with women with HER-2/neu-negative disease (Dawood et al., 2010). Trastuzumab toxicity mainly consists of the induction of congestive heart failure, which occurs only in a very small percentage of patients (Suter et al., 2007). However, the majority of patients with MBC who initially respond to Trastuzumab develop resistance within one year of commencing treatment (Nahta et al., 2006), which is known as acquired resistance. A subgroup of patients displays primary resistance to the level where the disease never responds to Trastuzumab.

Although continuation of Trastuzumab treatment beyond progression results in disease response or stability for some patients (Cortes et al., 2012), an alternative anti-HER-2/neu humanized mAb, which was named Pertuzumab, was later described by Franklin et al. (2004). By binding to the extracellular subdomain II of the HER-2 receptor (Figure-1), which is farther from the cell membrane, Pertuzumab prevents the oncogene from forming dimers with other HER receptors (or with other HER-2 receptors). It is the first member of a new class of targeted therapies known as HER-2 dimerization inhibitors (Agus et al., 2005; Adams el al., 2006). The binding of Pertuzumab to the dimerization arm of the HER-2/neu receptor sterically inhibits the ligand-dependent formation of HER-2-containing ErbB heterodimers (Franklin et al., 2004), most notably the highly mitogenic HER-2/HER-3 heterodimer (Lee-Hoeflich et al., 2008), and thereby inhibits ligand-initiated intracellular signalling events that are associated with tumor growth and progression.

Although of high therapeutic efficacy, the passive administration of mAbs suffers from some drawbacks including the need for frequent administration, the necessity for a prolonged duration, impossibility of application in a prophylactic manner in high-risk patients, cost intensiveness and targeting only single epitopes.

The aim of this study was to develop an anti-HER-2/neu vaccine composition for the treatment of an HER-2/neu-associated cancer, such as breast and gastric cancer.

To this end, the inventors have developed a multilevel approach to producing a vaccine composition by combining B cell epitopes and/or mimotopes with binding capacity to the anti-Her-2/neu antibodies trastuzumab and pertuzumab to induce broad coverage in active immunotherapy against HER-2/neu, together with a Th1-driving adjuvant to induce a broader and more effective anti-cancer responses. Moreover, combining these epitope/mimotope-based vaccines with the blockade of immunological checkpoints can help to reduce immune-suppressive pathways and support induction of efficacious antitumor responses.

This multilevel approach to producing as vaccine composition comprised the following:
1. Establishment of an epitope/mimotope platform using bioPeptide libraries.
2. Identification of pertuzumab- and trastuzumab-binding peptide fragments from the library;
3. In vitro and in vivo evaluation of the identified peptide sequences as single peptides or as a mixture;
4. Evaluation of immune responses against the mixture of the mimotopes in conjunction with potent Th1-promoting-adjuvants; and
5. Testing and evaluation of immune responses to the vaccine candidates (combined as a fusion protein) when co-applied with anti-CTLA-4 and/or anti-PD1 antibodies such as ipilimumab and tremelimumab.

### B Cell Epitopes

As described elsewhere herein, peptide sequences may be derived from known B cell epitopes of antigens associated with cancer or checkpoint antigens, or computer models and algorithms can be applied to predict B cell epitopes. However, computer models may not fully predict the exact binding site of the screening antibody and the natural epitope, and such predicted peptides need to be examined experimentally. Libraries of random and commercially available peptides, displayed on phages (Smith, 1985), are also widely used for identification of biologically relevant peptides. Using a phage displaying libraries of such random peptides, trastuzumab-binding epitopes and/or mimotopes with distinctive mimicry with B cell epitopes of HER-2/neu can be detected and used for immunization of mice to induce HER-2/neu specific antibodies. However, random peptides must be sufficiently large and degenerate in order to cover all or at least a significant proportion of possibilities. Additionally, in case two recognition sites for a proper binding of the screening antibody are required, peptides generated using the above technologies may not be detected by the antibody as the two recognition sites are on separate peptides. Such disadvantages can be over-come when focused and over-lapping peptides are used.

### Identification of Mimotopes Using Over-Lapping Peptides

Libraries can be synthesized in a systematic way applying DNA synthesis when the sequence of the protein is known. The PepID technology (ATG:biosynthetics, Germany, GmbH), which has been utilized in diagnostics (Van Regenmortel, 2009) as well as in therapeutic cancer therapy (Kenter et al., 2008), allows for the generation of such overlapping BioPeptide libraries. While random approaches mix strings of DNA, whether they are biologically relevant or not, in BioPeptide libraries as generated by PepID technology, a rational design approach is used to partition any known protein or protein-coding DNA into fragments with uniform lengths. Moreover, when the specific region of the protein of interest where the mimotopes are localized is known, certain biologically irrelevant amino acid stretches can be left out and the focus can be on the important structures. The over-lapping scheme of peptides generated by this technology, unlike the random peptides, will fully detect the peptides if the recognition site of the Ab or the mAb are located on two peptides (see FIG. 2).

The advantages of the PepID technology are as follows:
1. A focused generation of a library with non-random and over-lapping peptides.
2. All the generated over-lapping peptides are relevant to only the protein of interest, in contrast to random peptides.
3. There are no false positive clones with the generated over-lapping peptides.
4. There is an infinite access to the over-lapping peptides, so the library can be re-generated using a maintenance vector.
5. The length of the peptides is not limited.
6. The over-lapping peptides can be expressed as phage-display libraries (for mapping of immune response) or alternatively for epitope mapping of known antibody.

### Example 1: Generation and Identification of Mimotopes with Trastuzumab and Pertuzumab-Binding Capacity, for Immuno-Therapeutic Activity

The PepID technology is applied to generate BioPeptide libraries consisting of over-lapping peptides/mimotopes with pertuzumab- and trastuzumab-binding capacity. Applying this technology will increase the likelihood of identifying those mimotopes with the highest binding capacity to pertuzumab and trastuzumab. As depicted in FIG. 2, the generated over-lapping peptides/mimotopes are first cloned into a maintenance vector which serves as a source vector and can be propagated in E. coli. This means that the over-lapping peptides/mimotopes can be regenerated from the maintenance vector and/or the permanent stocks at any time and in almost unlimited quantity, a great advantage which stands out in contrast to random peptides. For production of the library of over-lapping peptides, the maintenance vector is digested, allowing the potential to individually obtain each of the over-lapping peptides/mimotopes (FIG. 2). Expression vectors containing a gene for a reporter protein (Rp) and one over-lapping peptide/mimotope will be constructed, and resulted expression library will be transformed into E. coli to yield individual clones for each over-lapping peptide/mimotope. Upon induction, each over-lapping peptide/mimotope will be expressed in fused form to the reporter gene product. Using immunological assays such as colony blot (Tobias et al., 2010), the E. coli clones each expressing one over-lapping peptide/mimotope will be examined, the expression vector from the positive clones will be extracted and sequencing will be applied to identify the sequence of the mimotope from the positive E. coli clone.

### Example 2: Use of a Potent Th1 Driving Adjuvant System to Enhance and Direct Immune Responses to HER-2/Neu

Adjuvants are employed in therapeutic vaccines targeting tumor antigens as a means to enhance T cell immunity, in particular, Th1 responses and IFN-γ production along with high antibody responses. Adjuvants can also overcome various tolerance mechanisms and facilitate induction of CTLs that can traffic to and lyse malignant cells and additionally effect angiogenesis. In this project, three adjuvants with Th1-promoting properties will be tested in conjunction with the identified mimotopes.

The adjuvant QS-21 (a saponin molecule) or AS01 is combined with the anti-HER-2/neu vaccine composition produced by the methods disclosed herein vaccine in order to augment the immune response against HER-2/neu.

Secondly, the adjuvant system ASO4, which is a liposome-based adjuvant that contains QS-21, as well as 3D-monophosphoryl lipid A (MPL), a nontoxic derivative of lipopolysaccharide from Salmonella Minnesota and a TLR4 agonist (Gargon et al., 2007), is examined.

The third adjuvant to be investigated in this project is Montanide, an emulsion which is increasingly used as adjuvant in new cancer vaccine candidates due to its capacity to induce both humoral and cellular immune responses.

### Example 3: Blockade of the Checkpoint CTLA-4 or PD1

Immune-checkpoint receptors include cytotoxic T-lymphocyte-associated antigen 4 (CTLA4) and PD1, which down-modulate the amplitude of T cell activation (Gelao et al., 2014). The vast majority of in vitro and in vivo studies on CTLA4 support its negative role in T cell activation contributing to the physiologic regulation of the immune response. Preclinical findings have shown that if there is an endogenous anti-tumour immune response in the animals after tumor implantation, blockade of such immune-checkpoints could enhance that endogenous response, which ultimately can induce tumor regression (Pardoll, 2012). These resulted in production and testing fully humanized anti-CTLA4 and anti-PD1 antibodies (Pardoll, 2012; Gelao, 2014). Immune-checkpoint inhibitor anti-PD1 is included with the vaccine composition produced by the methods disclosed herein, together with a selected Th1 adjuvant system.

### Example 4—Mimotope Mapping of Pertuzumab and Trastuzumab

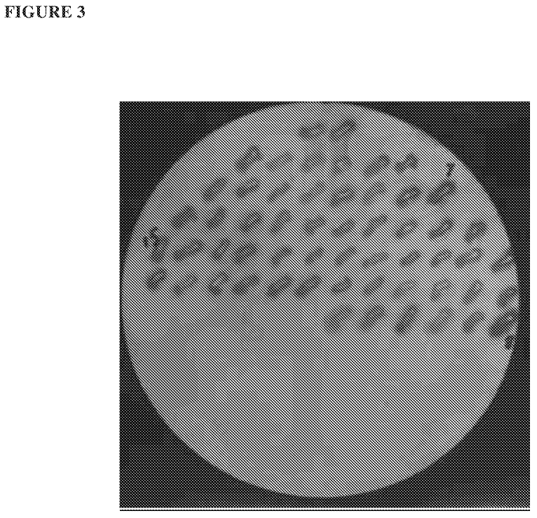

Based on the available protein sequence of subdomain II and IV of HER-2/neu (Fiore 1987), synthetic double-stranded DNA fragments encoding the over-lapping peptides will be generated by ATG:biosynthetics, Germany, GmbH. Concatamers of the over-lapping peptides will first be cloned into a maintenance vector, which can be stored, propagated and used an unlimited number of times as needed (FIG. 3). For production of a library with the overlapping peptides/mimotopes, the maintenance vector will be designed and each over-lapping peptide will be inserted into the fusion pEPX1 expression vector in which the peptides are as extensions of 26 kDa glutathione-S-transferase from Schistosoma japonicum (Sj26GST). The constructed pEPX1 vectors will be used to electroporate commercially available E. coli host strains. A portion of the electroporated bacteria will be used to prepare a library stock.

For epitope mapping of Pertuzumab and Trastuzumab using the generated library, colony blot assay will be applied (Tobias et al., 2010). A portion of the frozen stock of the library will be plated on selective plates to obtain single colonies. The obtained colonies will be streaked onto duplicate LB agar plates. One plate will be used for the colony blot assay, using nitrocellulose membrane, which will be treated according to standard methods for lysis of the cells and preparation for detection of clones binding to the monoclonal antibodies. The detection of binding will then be carried out using an appropriate secondary antibody conjugated to an enzyme allowing detection of the colonies to which the mAb has been bound. Positive colonies will be picked from the matched duplicate plate, grown and plasmids will be prepared. The sequences of the inserts in the individual plasmids will be determined by sequencing from the 26SjGST gene. All sequencing will be outsourced and performed at Eurofins MWG DNA (Germany).

### Example 5—Animal Studies

### Coupling of the Identfied Mimotopes

The peptide sequences identified from the library, as a mixture of single peptides or as a fusion thereof, will be end-linked and coupled to the well characterized carrier protein CRM-197 which has been used for conjugate vaccines against Neisseria meningitidis and Streptococcus pneumoniae (Lagos et al., 1999). The coupling of the mimoptoes to CRM-197 will be outsourced and performed at PiChem, Graz, Austria.

mice

Eight-week-old female BALB/c mice will be purchased from Charles River (Sulzfeld, Germany). All experiments will have an approval by the Animal Experimentation Committee of the University of Vienna and Ministry of Education, Science and Culture.

### Immunization

Mice (5 mice/group) will be immunized with the single coupled-peptides (25 μg/mouse) or as a fusion peptide along with the Th1-driving adjuvants with or without the inhibitor of immune-checkpoint anti-PD1. The control group will receive CRM-197 and adjuvants alone. The immunizations will be carried out 4 times in 21 day intervals. Seven days after the last immunization the animals will be sacrificed. Immunizations with the coupled mimotopes, will be carried out in rabbits at the laboratories of Charles River (Kissleg, Germany). According to the immunization schedule previously tested in mice, these immunizations will be done 4 times at 14-21 day intervals.

### Sampling

Blood samples from mice will be taken by tail bleeding before immunization and seven days after the last immunization. Blood samples from the rabbit will be taken by puncture of an ear-vein before and after immunization with the peptide mixture. Spleens, hearts, livers, kidneys and lungs from mice will be removed for histopathological analyses.

### ELISA for Specific Antibody Responses

Enzyme-linked immunosorbent assays (ELISA) will be carried out to detect peptide-specific antibody (Ab) responses. Microtiter plates will be coated with the examined peptide sequences. As the peptides will be coupled to CRM-197, peptides coupled to unrelated linker will be used for coating. After blocking the non-specific sites, diluted sera of the mice immunized with the same peptide sequences will be added to the antigen-coated plates. The unbound sera will be removed by washing, followed by adding an appropriate secondary anti-mouse Ig conjugated to a detection system (e.g., HRP). HER-2/neu specific Ab responses, using intact Her-2/neu as coating antigen. Microtiter plates will be coated with either Pertuzumab or Trastuzumab, and after blocking the non-specific sites the plates will be incubated with microsomal preparation of SK-BR-3 as a source for intact HER-2/neu diluted in PBS (Jasinska et al., 2003). After washing and blocking, diluted mouse antisera will be added to the plates followed adding an appropriate secondary anti-mouse Ig conjugated to a detection system (e.g., HRP). HER-2/neu specific Ab responses, using chimera HER-2/neu. As both Pertuzumab and Trastuzumab bind to the ECD of HER-2/neu, a chimera of Her-2/neu (RD Systems) consisting of extracellular domain of Her-2/neu will also be used for coating. Microtiter plates will be coated with either Pertuzumab or Trastuzumab, and after blocking the non-specific sites the plates will be incubated with the HER-2/neu chimera diluted in PBS. After washing and blocking, diluted mouse antisera will be added to the plates followed adding an appropriate secondary anti-mouse Ig conjugated to a detection system (e.g., HRP).

### Proliferation Inhibition

The biological effects of the induced Abs against the peptide sequences and the antitumor effectiveness (i.e., tumor growth inhibition) by these Abs will be assessed in vitro, using a well-established [³H]-thymidine proliferation assay (Jasinska et al., 2003). The SK-BR-3 tumor cell will be incubated with mice sera, or Trastuzumab or Pertuzumab as controls, followed by pulsing the cells with thymidine. Percentage of inhibition of proliferation will be calculated by comparing the cpm (counts per minute) values of treated cells with those of non-treated cells.

### T Cell Proliferation and Cytokine Production in Spleen Cell Culture

Spleen cells from peptide-sensitized and control animals, activated only with the conjugation partner CRM-197, (the B cell epitopes or mimotopes cannot be used for T cell proliferation assays) will be used to detect T cell proliferation (Hafner et al., 2005). The supernatant of activated spleen cells will also be used to measure the level of cytokine (e.g., IFN-g, IL-2) production (Hafner et al., 2005).

### Antibody Dependent Cell-Mediated Cytotoxicity (ADCC and Complement Dependent Cytotoxicity (CDC) Assays

IgG from sera of immunized mice will be purified, and used for CDC and ADCC as described (Jasinska et al., 2003).

By combining peptide sequences with binding capacity not only to Trastuzumab but also to Pertuzumab together with a proper Th1-driving adjuvant and a blockade of immune-checkpoint, the proposed multilevel approach disclosed herein will produce an anti-HER-2/neu vaccine composition against breast, gastric, and other HER-2 overexpressing cancer types.

### Example 6—Generation and Identification of Mimotopes with Trastuzumab and Pertuzumab-Binding Capacity and the Capacity of Mimotopes Fusion Proteins to Inhibit her-2-Positive Tumour Growth In Vivo

### A. Generation and Expression of Overlapping Peptides

Protein sequences corresponding to the extracellular domains of Her-2/neu (P04626; Uniprot) were electronically back-translated into DNA sequences, partitioned into uniformly-sized overlapping peptides, and used for individual insertion into the expression vector pEPX-1 (ATG: Biosynthetics), as described (Tobias et al, 2018; Submitted), and pools with the expression vectors were used as libraries for expression in E. coli BL21 (Tobias et al, 2018; Submitted).

### B. Detection of Mimotopes and Sequencing

Bacterial colonies individually expressing an overlapping peptide were screened by colony blot assay, and cultures of each clone expressing a mimotope were lysed using lysis buffer (Tris HCl pH 8.0 50 mM, 1 mg/ml lysozyme) for use in dot blot assay, as described (Tobias et al, 2018; Submitted), using the mAbs Trastuzumab (200 μg/ml) or Pertuzumab (30 μg/ml).

Bacteria from clones corresponding to positive colonies found in the colony blot assay were selected from the master plates and prepared for sequencing as described (Tobias et al, 2018; Submitted).

### C. Peptide Synthesis

Sequences of the identified mimotopes of Trastuzumab (JTMH1 and JTMH2) and Pertuzumab (JTMP) were sent for synthesis at piChem (Austria).

### D. Peptides for EMISA or Immunizations

The following peptides, in free or CRM197-conjugated forms, were used for immunization experiments or for use in ELISA, respectively:

JTMP:
(SEQ ID NO: 2)
HSGICELHCPALVTYNTDTFESMPNPEGRYTFGASCVTACPY;
ITMH1:
(SEQ ID NO: 3)
CSQFLRGQECVEECRVLQGLPREYVNARHCLPCHPECQPQNGSVTCFGPS
JTMH2:
(SEQ ID NO: 4)
GVKPDLSYMPIWKFPDEEGACQPCPINCTHSCVDLDDKGCPAEQRASPLT

### E. Immunization Experiments

Female BALB/C mice (Charles River, Sulzfeld. Germany; 6-8 week of age at the time of delivery) were used in subcutaneous immunization studies. Three immunizations were given in 3 weeks intervals, and blood samples were taken prior each immunization and three weeks after the last immunization when the mice were sacrificed.

A transgenic mouse model was applied for evaluating the anti-tumor effect of the examined mimotopes:

Female FVB/N mice transgenic for the activated rat c-neu oncogene (MMTV-c-neu, 5- to 9-week-old; Charles River, Sulzfeld, Germany) were used. Overexpression of the c-neu oncogene is driven by a mouse mammary tumor virus (MMTV) promoter and these mice transgenic for the activated rat c-neu oncogene develop spontaneously mammary tumors by ˜30 weeks of age [Wagner et al, Breast Cancer Res Treat, 2007, 106:29-38]. Mammary glands were inspected weekly for tumor appearance and progression. Tumors were measured with a caliper and the volume was calculated by: x2 y/2, whereby x and y represent the short and long dimensions of the tumor. Total tumor volume per mouse was calculated by adding all tumor volumes. Progressively growing masses of >3 mm·3 mm were regarded as tumors. Mice were sacrificed for ethical reasons at the time when a total tumor volume of approximately 2,000 mm3 was exceeded. Tumors were excised and used for histological analyses.

### F. Detection of Peptide-Specific Serum IgG

Microtiter plates (Nunc Maxisorp, Denmark) were coated with uncoupled peptides, in carbonate buffer (0.5 μg/well), and ELISA was performed as previously described in Example 5, above. After blocking, diluted sera from the immunized mice were added. Bound IgG were detected with HRP-labelled rabbit anti mouse IgG antibody and subsequent TMB staining. For detection of IgG2a isotype, rat anti mouse IgG2a (BD Biosciences, USA) and the secondary antibody HRP-labelled mouse anti-rat IgG (Jackson Immuno Research, USA) were used, followed by TMB staining. Plates were read after adding stop solution at 450 vs 630 nm.

G. Detection of her-2/Neu-Specific IgG

A fusion protein consisting of the recombinant extracellular domain of human Her-2/neu (amino acid residues 23-652) fused to Fc region of human IgG1 (ErbB2/Fc Chimera, R&D Systems) was used as coating antigen. Plates were coated with 0.1 μg/well, and detection of Her-2/neu specific IgG antibodies was carried out as described above.

### H. Cytokines Production in Cultures of Splenocytes

Splenocytes of the sacrificed mice were taken aseptically, minced, sterile-filtered and cell suspensions were prepared. Cells (5×10⁵ per well) were plated in 96-well round-bottomed plates, and stimulated for 72 h in culture medium (RPMI 1640, with 10% heat-inactivated FCS, 2 mM L-Glutamine) at 37° C., 95% humidity and 5% CO₂. Supernatants were harvested and stored at −20° C., until analysis. Levels of secreted IL-2, IFNγ and IL-5 were measured by ELISA according to manufacturer's instructions (Affymetrix eBioscience, USA), and expressed in pg/ml.

### Results

### I. Identification and In Vitro Characterization of Trastuzumab and Pertuzumab Mimotopes:

### J. Pertuzumab Mimotope

For identification of Pertuzumab mimotopes, a library of 15-mer overlapping peptides spanning the extracellular domain of Her-2 (ECD-II) was used for electroporation into E. coli BL21. A number of colonies, each expressing one overlapping peptide, were picked and examined by colony blot assay (FIG. 3). Sequence analysis on plasmids from the clones giving the highest signal in the colony blot assay showed four overlapping peptides with sequences reported to be in the binding region of Pertuzumab (as described in Deng et al, Plos One, 2014, 5; 9(9):e106448).

| Identifed peptides | Signal by | Position |
|---|---|---|
| (15-mer) | colony blot | in ECD-II |
| HSGICELHCPALVTY | Strong | 238-252 |
| PALVTYNTDTFESMP | Strong | 247-261 |
| SMPNPEGRYTFGASC | Strong | 259-273 |
| GRYTFGASCVTACPY | Strong | 265-279 |

The identified peptides are within the region of Her-2 ECD-II reported as the binding site epitope of Pertuzumab by Deng et al. (2014). See also Franklin et al, Cancer Cell, 2004; Cancer Cell.; 5(4):317-28).

A 42-mer peptide harboring the four detected 15-mer peptides was assigned as the mimotope of Pertuzumab and designated as JTMP (SEQ ID NO:2):

HSGICELHCPALVTYNTDTFESMPNPEGRYTFGASCVTACPY (amino
acid residues 238-279 of Her-2).

### K. Trastuzumab Mimotopes

A library of 15-mer overlapping peptides spanning the ECD-IV of Her-2 was used for electroporation into E. coli BL21, followed by screening of clones. However, no significant positive clone was detected by the colony blot assay. As it has been reported that the binding epitope of Trastuzumab is discontinuous (see, e.g., Cho et al., Nature, 2003 Feb. 13; 421(6924):756-60), a new library of the 50 aa overlapping peptides also spanning the ECD-IV of Her-2 was examined.

Following examining the new library (including 3 tubes each with 1-2 pEPX-1 vectors), positive clones were detected and sequence analyses on the plasmid of these clones revealed overlapping peptides which have been reported to be the binding epitope of Trastuzumab.

| | | Amino acid | |
|---|---|---|---|
| Identified | Signal by | position in | |
| peptides (50-mer) | colony blot | Her-2 ECD-IV | Designation |
| CSQFLRGQECVEECRVLQGL | Strong | (509-558) | JTMH1 |
| PREYVNARHCLPCHPECQP | | | (SEQ ID |
| QNGSVTCFGPE | | | NO: 3) |
| GVKPDLSYMPIWKFPDEEG | Strong | (581-630) | JTMH2 |
| ACQPCPINCTHSCVDLDDKG | | | (SEQ ID |
| CPAEQRASPLT | | | NO: 4) |

Based on the above results we decided to use the two sequences, from the clones which had given the strongest signal, as Trastuzumab mimotopes JTMH1 and JTMH2, respectively.

### REFERENCES

Adams C W, Allison D E, Flagella K, el al. Humanization of a recombinant monoclonal antibody to produce a therapeutic HER dimerization inhibitor, pertuzumab. Cancer zaImmunol Immunother 2006; 55:717-27
Agnandji S T, Lell B, Fernandes J F, Abossolo B P, Methogo B G, Kabwende A L, Adegnika A A, Mordmuller B, Issifou S, Kremsner P G, et al. A phase 3 trial of RTS,S/AS01 malaria vaccine in African infants. N. Engl. J. Med. 2012; 367:2284-2295
Agus D B, Gordon M S, Taylor C, et al. Phase I clinical study of pertuzumab, a novel HER dimerization inhibitor, in patients with advanced cancer. J Clin Oncol 2005; 23:2534-43
Baselga J, Corte's J, Kim S-B, et al. Pertuzumab plus trastuzumab plus docetaxel for metastatic breast cancer. N Engl J Med 2012; 366:109-19
Bray F, McCarron P, Parkin D M. The changing global patterns of female breast cancer incidence and mortality Breast Cancer Res 2004; 6:229-239
Burgess A W, Cho H S, Eigenbrot C, Ferguson K M, Garret T P J, Leahy D J, Lemmon M A, Sliwkowski M X, Ward C W, Yokoyama S. An Open-and-Shut Case? Recent Insights into the Activation of EGF/ErbB Receptors. Molecular Cell, 2003; 12:541-552
Cortes J, Fumoleau P, Bianchi G V, et al. Pertuzumab monotherapy after trastuzumab-based treatment and subsequent reintroduction of trastuzumab: activity and tolerability in patients with advanced human epidermal growth factor receptor 2-positive breast cancer. J Clin Oncol 2012; 30:1594-600
Dawood S, Broglio K, Buzdar A U, Hortobagyi G N, Giordano S H. Prognosis of Women With Metastatic Breast Cancer by HER-2 Status and Trastuzumab Treatment: An Institutional-Based Review. J Clin Oncol 2010; 28:92-98
Decker T, Fischer G, Bucke W, Bucke P, Stotz F, Gruneberger A, Gropp-Meier M, Wiedemann G, Pfeiffer C, Peschel C et al Increased number of regulatory T cells (T-regs) in the peripheral blood of patients with Her-2/neupositive early breast cancer. J Cancer Res Clin Oncol 2012; 138:1945-1950
Disis, M L, Pupa S M, Gralow J R, Dittadi R, Menard S, Cheever M A. High-titer HER-2/neu protein-specific antibody can be detected in patients with early-stage breast cancer. J Clin Oncol 1997; 15:3363-3367
Disis M L, Gooley T A, Rinn K, Davis D, Piepkorn M, Cheever M A, Knutson K L, Schiffman K. Generation of T Cell Immunity to the HER-2/neu Protein After Active Immunization With HER-2/neu Peptide-Based Vaccines. J Clin Onco 2002; 20:2024-2032
Dranoff G. Cytokines in cancer pathogenesis and cancer therapy. Nat. Rev. Can. 2004. 4:11-22
Eiermann W; International Herceptin Study Group. Trastuzumab combined with chemotherapy for the treatment of HER-2-positive metastatic breast cancer: pivotal trial data. Ann Oncol. 2001. 12 Suppl 1:S57-62
Fiore P D. 1987. ErbB-2 is a potent oncogene when overexpressed in NIH/3T3 cells. Science 1987; 237:178-182
Franklin M C, Carey K D, Vajdos F F, et al. Insights into ErbB signaling from the structure of the ErbB2-pertuzumab complex. Cancer Cell 2004; 5:317-328
Garçon N, Chomez P, Van Mechelen M. GlaxoSmithKline Adjuvant Systems in vaccines: concepts, achievements and perspectives. Expert Rev Vaccines 2007; 6:723-39
Garg A, Li J, Clark E, Knott A, Carrothers T J, Marier J F, Cortés J, Brewster M, Visich J, Lum B. Exposure-response analysis of pertuzumab in HER-2-positive metastatic breast cancer: absence of effect on QTc prolongation and other ECG parameters. Cancer Chemother Pharmacol 2013; 72:1133-1141
Gelao L, Criscitiello C, Esposito A, Goldhirsch A, Curigliano G. Immune Checkpoint Blockade in Cancer Treatment: A Double-Edged Sword Cross-Targeting the Host as an “Innocent Bystander”. Toxins 2014; 6:914-933
Generali D, Bates G, Berruti A, Brizzi M P, Campo L, Bonardi S, Bersiga A, Allevi G, Milani M, Aguggini S, Dogliotti L, Banham A H, Harris A L, Bottini A, Fox S B. Immunomodulation of FOXP3+ regulatory T cells by the aromatase inhibitor letrozole in breast cancer patients. Clin Cancer Res 2009; 15:1046-1051
Geysen H M, Rodda S J, Mason T J. A priori delineation of a peptide which mimics a discontinuous antigenic determinant, Molecular immunology 1986; 23:709-15
Gravalos C, Jimeno A. HER2 in gastric cancer: a new prognostic factor and a novel therapeutic target. Ann Oncol 2008; 19:1523-1529
Hafner C, Wagner S, Jasinska J, Allwardt D, Scheiner O, Wolff K, Pehamberger H, Wiedermann U, Breiteneder H. Epitope-specific antibody response to Mel-CAM induced by mimotope immunization. J Invest Dermatol. 2005; 124:125-31
Hanahan D, Weinberg R A. Hallmarks of Cancer: the next generation. Cell 2011; 144: 646-674
Jasinska J, Wagner S, Badauer C, Sedivy R, Brodowicz T, et al. Inhibition of tumor cell growth by antibodies induced after vaccination with peptides derived from the extravellular domain of HER-2/neu. Int J Cancer 2003; 107:976-983
Jensen-Jarolim E, Singer J. Cancer vaccines inducing antibody production: more pros than cons Expert Rev. Vaccines 2011; 10:1281-1289
Kenter G G et al. (2008). Cancer Therapy: Clinical: Phase I Immunotherapeutic Trial with Long Peptides Spanning the E6 and E7 Sequences of High-Risk Human Papillomavirus 16 in End-Stage Cervical Cancer Patients Shows Low Toxicity and Robust Immunogenicity Clin Cancer Res 2008; 14:169-177
Ladjemi M Z, Jacot W, Chardes T, Pelegrin A, Navarro-Teulon I. Anti-HER-2 vaccines: new prospects for breast cancer therapy. Cancer Immunol Immunother 2010; 59:1295-1312
Lagos R, Munoz A, Levine M M, Watson W, Chang I, Paradiso P. Immunology of combining CRM(197) conjugates for Streptococcus pneumoniae, Neisseria meningitis and Haemophilus influenzae in Chilean infants. Vaccine 2009; 14:2299-2305
Livingston P O, Adluri S, Helling F, Yao T J, Kensil C R, Newman M J, Marciani D. Phase 1 trial of immunological adjuvant QS-21 with a GM2 ganglioside-keyhole limpet haemocyanin conjugate vaccine in patients with malignant melanoma. Vaccine. 1994; 12:1275-1280
Liu J K H. Anti-Cancer Vaccines—A One-Hit Wonder? Yale Journal Of Biology and Medicine 2014; 87:481-489
Maeda K, Hazama S, Tokuno K, Kan S, Maeda Y, Watanabe Y, Kamei R, Shindo Y, Maeda N, Yoshimura K, Yoshino S, Oka M. Impact of chemotherapy for colorectal cancer on regulatory T cells and tumor immunity. Anticancer Res 2011; 31:4569-4574
Marty M, Cognetti F, Maraninchi D, et al. Randomized phase II trial of the efficacy and safety of trastuzumab combined with docetaxel in patients with human epidermal growth factor receptor 2-positive metastatic breast cancer administered as first-line treatment: the M77001 study group. J Clin Oncol 2005; 23:4265-74
Moore Al, McCarthy L, Mills K H. The adjuvant combination monophosphoryl lipid A and QS21 switches T cell responses induced with a soluble recombinant HIV protein from Th2 to Th1. Vaccine. 1999; 17:2517-2527
Nahta R, Hung M-C, Esteva F J. The HER-2-targeting antibodies trastuzumab and pertuzumab synergistically inhibit the survival of breast cancer cells. Cancer Res 2004; 64:2343-6
Nahta R, Yu D, Hung M C, Hortobagyi G N, Esteva F J. Mechanisms of disease: understanding resistance to HER-2-targeted therapy in human breast cancer. Nat Clin Pract Oncol 2006; 3:269-80
Pardoll D. The blockade of immune checkpoints in cancer immunotherapy Nature 2012; 12: 252-264
Pupa, S. M., S. Menard, S. Andreola, and M. I. Colnaghi. Antibody response against the c-erbB-2 oncoprotein in breast carcinoma patients. Cancer Res 1993; 53:5864-5866
Riemer A B, Klinger M, Wagner S, Bernhaus A, Mazzucchelli L, Pehamberger H, Scheiner O, Zielinski C C, Jensen-Jarolim E. Generation of Peptide mimics of the epitope recognized by trastuzumab on the oncogenic protein Her-2/neu. J Immunol. 2004; 173:394-401
Riemer A B, Jensen-Jarolim E. Mimotope vaccines: Epitope mimics induce anti-cancer Antibodies. Immunol Lett 2007; 113:1-5
Ross J S, Slodkowska E A, Symmans W F, Pusztai L, Ravdin P M, Hortobagyi G N. The HER-2 receptor and breast cancer: ten years of targeted anti-HER-2 therapy and personalized medicine. Oncologist 2009; 14:320-68
Rüschoff J, Hanna W, Bilous M, Hofmann M, Osamura R, Penault-Llorca F, van de Vijver M, Viale G. HER2 testing in gastric cancer: a practical approach. Mod Pathol 2012; 25:637-650
Schlessinger J, Ullrich A. Growth factor signalling by receptor tyrosine kinases. Neuron 1992; 9:383-391
Shurin M R, Lu L, Kalinski P, Stewart-Akers A. M, Lotze M T. Th1/Th2 balance in cancer, transplantation and pregnancy Springer Semin Immunopathol 1999; 21:339-359
Slamon D J, Clark G M, Wong S G, Levin W J, Ullrich A, McGuire W L. Human breast cancer: correlation of relapse and survival with amplification of the HER-2/neu oncogene. Science 1987; 235: 177-182
Slamon D J, Leyland-Jones B, Shak S, Fuchs H, Paton V, Bajamonde A, Fleming T, Eiermann W, Wolter J, Pegram M, Baselga J. Norton L. Use of Chemotherapy plus a Monoclonal Antibody against HER2 for Metastatic Breast Cancer That Overexpresses HER2. N Engl J Med 2001: 344:783-792
Smith G P. Filamentous fusion phage: Novel expression vectors that display cloned antigens on the virion surface. Science. 1985; 228:1315-1317
Smyth M J, Ngiow S F, Teng M W L. Targeting regulatory T cells in tumor immunotherapy. Immunology and Cell Biology 2014; 92:473-474
Suter T M, Procter M, van Veldhuisen D J, Muscholl M, Bergh J, Carlomagno C, Perren T, Passalacqua R, Bighin C, Klijn J G M, Ageev F T, Hitre E, Groetz J, Iwata H, Knap M, Gnant M, Muehlbauer S, Spence A, Gelber R D, Piccart-Gebhart M J. Trastuzumab-associated cardiac adverse effects in the Herceptin adjuvant trial. J Clin Oncol 2007; 25:3859-3865
Swain S M, Ewer M S, Corte's J, Amadori D, Miles D, Knott A, Clark E, Benyunes M C, Ross G, Baselga J. Cardiac tolerability of pertuzumab plus trastuzumab plus docetaxel in patients with HER-2-positive metastatic breast cancer in CLEOPATRA: a randomized, double-blind, placebo-controlled phase III study. Oncologist. 2013; 18:257-264
Tobias J, Holmgren J, Heilman M, Nygren E, Lebens M, Svennerholm A M. Over-expression of major colonization factors of enterotoxigenic Escherichia coli, alone or together, on non-toxigenic E. coli bacteria. Vaccine. 2010; 28:6977-6984
Vogel C L, Cobleigh M A, Tripathy D, Gutheil J C, Harris L N, Fehrenbacher L, Slamon D J, Murphy M, Novotny W F, Burchmore M, Shak S, Stewart S J, Press M. Efficacy and safety of trastuzumab as a single agent in first-line treatment of HER-2-overexpressing metastatic breast cancer. J Clin Oncol 2002; 20:719-726
Wagner S, Hafner C, Allwardt D, Jasinska J, Ferrone S, Zielinski C C, Scheiner O, Wiedermann U, Pehamberger H, Breiteneder H. Vaccination with a human high molecular weight melanoma-associated antigen mimotope induces a humoral response inhibiting melanoma cell growth in vitro. J Immunol. 2005; 174:976-82
Wagner S, Jasinska J, Breiteneder H, Kundi M, Pehamberger H, Scheiner O, Zielinski C C, Wiedermann U. Delayed tumor onset and reduced tumor growth progression after immunization with a Her-2/neu multi-peptide vaccine and IL-12 in c-neu transgenic mice. Breast Cancer Res Treat 2007; 106:29-38
Wagner S, Krepler C, Allwardt D, Latzka J, Strommer S, Scheiner O, Pehamberger H, Wiedermann U, Hafner C, Breiteneder H. Reduction of human melanoma tumor growth in severe combined immunodeficient mice by passive transfer of antibodies induced by a high molecular weight melanoma-associated antigen mimotope vaccine. Clin Cancer Res. 2008; 14:8178-83
Ward R L, Hawkins N J, Coomber D, Disis M L. Antibody immunity to the HER-2/neu oncogenic protein in patients with colorectal cancer. Hum Immunol 1999; 60:510-515
Wiedermann U, Wiltschke, Jasinska, Kundi, Zurbriggen, Garner-Spitzer E, Bartsch R, Steger G, Pehamberger H, Scheiner O, Zielinski C C. A virosomal formulated Her-2/neu multi peptide vaccine induces Her-2/neu-specific immune responses in patients with metastatic breast cancer: a phase I study. Breast Cancer Res Treat 2010; 119:673-683
Wiedermann U, Davis A B, Zielinski C C. Vaccination for the prevention and treatment of breast cancer with special focus on Her-2/neu peptide vaccines. Breast Cancer Res Treat. 2013; 138:1-12
Wong C R Okada C Y, Levy R. TCR vaccines against T cell lymphoma: QS-21 and IL-12 adjuvants induce a protective CD8+ T cell response. J Immunol 1999; 162:2251-2258
Yarden Y, Sliwkowski M X, Untangling the ErbB signaling network. Nat Rev Mol Cell Biol 2001; 2:127-137
Yip Y L, Smith G, Koch J, Dubel S, and Ward R L. Identification of Epitope Regions Recognized by Tumor Inhibitory and Stimulatory Anti-ErbB-2 Monoclonal Antibodies: Implications for Vaccine Design. The Journal of Immunology 2001; 166: 5271-5278

1. A method of producing a vaccine composition for the treatment of cancer comprising a fusion protein of at least two peptide sequences, the method comprising:
(a) generating a library of peptide sequences;
(b) obtaining at least one antibody that binds to an antigen associated with cancer and/or a checkpoint antigen;
(c) screening the library generated in step (a) with the at least one antibody of step (b) to identify at least two peptide sequences that specifically bind to the at least one antibody; and
(d) combining at least two of the peptide sequences identified in step (c) to produce a fusion protein,
wherein the fusion protein, when administered to a subject, induces an antibody response directed against the antigen associated with cancer and/or the checkpoint antigen.

2. The method of claim 1, wherein the fusion protein, when administered to a subject, induces an antibody response directed against the antigen associated with cancer and the checkpoint antigen.

3. The method of claim 1, wherein the library of peptide sequences generated in step (a) comprises fragments of the antigen associated with the cancer.

4. The method of claim 1, wherein the library of peptide sequences generated in step (a) comprises fragments of the checkpoint antigen.

5. The method of claim 1, wherein the library of peptide sequences generated in step (a) comprises adjacent and overlapping fragments of the antigen associated with cancer and/or the checkpoint antigen.

6. The method of claim 5, wherein the library of peptide sequences generated in step (a), when arranged as a continuous amino acid sequence, comprise the amino acid sequence of the extracellular domain of the antigen associated with cancer and/or the checkpoint antigen in its native reading frame.

7. The method of claim 5, wherein the library of peptide sequences generated in step (a) consists of adjacent and overlapping fragments of the extracellular domain of the antigen associated with cancer and/or the checkpoint antigen.

8. The method of claim 5, wherein the library of peptide sequences generated in step (a) comprises adjacent fragments that overlap by at least two amino acids.

9. The method of claim 8, wherein the library of peptide sequences generated in step (a) comprises adjacent fragments that overlap by at least three amino acids.

10. The method of claim 1, wherein the library of peptide sequences generated in step (a) comprises fragments of about 15 amino acids to about 50 amino acids in length.

11. (canceled)

12. The method of claim 1, wherein step (b) comprises obtaining two or more antibodies that bind to the antigen, and step (c) comprises screening the library of peptide sequences generated in step (a) with the two or more antibodies, wherein each of the two or more antibodies specifically binds to a different peptide sequence in the library generated in step (a).

13. (canceled)

14. (canceled)

15. The method of claim 1, wherein the library of peptide sequences generated in step (a) comprises two or more B cell epitopes of the antigen associated with cancer and/or two or more B cell epitopes of the checkpoint antigen.

16. The method of claim 1, wherein the library of peptide sequences generated in step (a) comprises at least one B cell epitope of the antigen associated with cancer and at least one B cell epitope of the checkpoint antigen.

17. The method of claim 1, wherein the library of peptide sequences generated in step (a) comprises at least one mimotope.

18. The method of claim 1, wherein step (a) comprises:
(i) providing two or more nucleic acid sequences encoding the peptide sequences;
(ii) inserting each of the nucleic acid sequences of step (i) into an expression vector to generate a library of expression vectors;
(iii) transforming host cells with each of the expression vectors of step (ii) to generate a population of transformed host cells;
(iv) establishing clonal colonies of the transformed host cells generated in step (iii) to yield individual clones for each expression vector, and
(v) culturing the clonal colonies established in step (iv) under conditions suitable to express the peptide sequences.

19. The method of claim 1, further comprising attaching the fusion protein to a carrier.

20. (canceled)

21. The method of claim 19, wherein the carrier is diphtheria toxin variant CRM-197 (SEQ ID NO:1).

22. The method of claim 1, wherein the composition further comprises an adjuvant.

23. (canceled)

24. The method of claim 1, wherein the antigen associated with cancer is selected from the group consisting of HER-2/neu, epidermal growth factor, HER-1, an antigen of the melanoma antigen-encoding gene A family, B melanoma antigen, carcinoembryonic antigen, glycoprotein 100, telomerase transcriptase and mucin-1.

25. The method of claim 1, wherein the fusion protein comprises an amino acid sequence selected from the group consisting of SEQ ID NOs:2 to 8.

26. The method of claim 1, wherein the fusion protein comprises an amino acid sequence selected from the group consisting of SEQ ID NOs:2, 3, and 4.

27. The method of claim 1, wherein the checkpoint antigen is selected from the group consisting of CTLA-4, PD-1, PD-L1, PD-L2, LAG-3, OX40, OX40L, TIM-3 and GAL-9.

28. The method of claim 1, wherein the composition further comprises a checkpoint inhibitor.

29. (canceled)

30. (canceled)


SEQUENCE LISTING (8 sequences)

SEQ ID NO: 1 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 42 aa, homo sapiens
HSGICELHCPALVTYNTDTFESMPNPEGRYTFGASCVTACPY

SEQ ID NO: 3 - protein, 50 aa, homo sapiens
CSQFLRGQECVEECRVLQGLPREYVNARHCLPCHPECQPQNGSVTCFGPE

SEQ ID NO: 4 - protein, 50 aa, homo sapiens
GVKPDLSYMPIWKFPDEEGACQPCPINCTHSCVDLDDKGCPAEQRASPLT

SEQ ID NO: 5 - protein, 15 aa, homo sapiens
HSGICELHCPALVTY

SEQ ID NO: 6 - protein, 15 aa, homo sapiens
PALVTYNTDTFESMP

SEQ ID NO: 7 - protein, 15 aa, homo sapiens
SMPNPEGRYTFGASC

SEQ ID NO: 8 - protein, 15 aa, homo sapiens
GRYTFGASCVTACPY